(19) United States
(12) Patent Application Publication
(10) Pub. No.: US 2025/0206843 A1
(43) Pub. Date: Jun. 26, 2025

(54) BINDING MOLECULES SPECIFIC TO HUMAN EPIDERMAL GROWTH FACTOR RECEPTOR 2

(71) Applicant: The National Institute for Biotechnology in the Negev Ltd., Beer Sheva (IL)

(72) Inventors: Niv PAPO, Raanana (IL); Lital BEN NAIM, Beer Sheva (IL)

(21) Appl. No.: 18/848,326

(22) PCT Filed: Apr. 2, 2023
(86) PCT No.: PCT/IL2023/050340

§ 371 Date: Sep. 18, 2024

Related U.S. Application Data
(60) Provisional application No. 63/326,922, filed on Apr. 4, 2022.

(51) Int. Cl. C07K 16/32; A61K 47/68; G01N 33/574

(52) U.S. Cl. CPC C07K 16/32; A61K 47/68033; A61K 47/6849; A61K 47/6855; G01N 33/57415; C07K 2317/22; C07K 2317/569; C07K 2317/77; C07K 2317/92; C07K 2319/21

(57) ABSTRACT

The present invention provides binding molecules, in particular VHH antibodies (nanobodies), that recognize HER2 protein with high affinity and specificity. The present invention further provides pharmaceutical compositions comprising the binding molecules and methods for their use in treating cancer.

### FIELD OF THE INVENTION

The invention is in the field of immunotherapy and relates to binding molecules, in particular single domain antibodies (e.g., nanobodies or VHH molecules) that specifically bind human epidermal growth factor receptor 2 (HER2). Conjugates comprising the binding molecules and compositions comprising them, for diagnosis and treatment of diseases, in particular cancer, are also included.

### BACKGROUND OF THE INVENTION

HER2 is a member of the Human epidermal growth factor receptors (HERs), a receptor tyrosine kinase (RTK) sub-family, which is a conserved family of four single-pass trans-membranal (TM) signaling receptors, HER1 (also referred to as EGFR/ErbB1), HER2 (ErbB2), HER3 (ErbB3) and HER4 (ErbB4). These HER protein isoforms are expressed in cells of mesodermal origin (musculoskeletal, vascular, urinogenital systems, connective tissues, and dermis) and ectodermal origin (epidermis and nerves). These receptor tyrosine kinases play a key role in regulation of cell signaling pathways affecting cell proliferation and migration (both important for tumorigenesis), and also adhesion, differentiation, and apoptosis.

HER receptors are frequently mutated and/or overexpressed in different types of human cancers. The complex formed by HER2 and HER3 is the most potent heterodimer in the HER family and is considered a major player and contributor to the progression of different types of cancers such as breast, gastric, ovarian, and colorectal cancer. These two TM receptors function as an oncogenic unit by forming a heterodimer through interactions between their extracellular dimerization arms (in subdomain II), TM, and c-terminal domains. HER2 exists in an open conformation exposing its dimerization domain, while HER3 is activated by ligand (NRG, Neuregulin-1) binding and then change its conformation from closed to open. When interactions form, HER2/HER3 initiates key signaling pathways and transcription factors regulating genes that affect various cellular functions, including the mitogen-activated protein kinase (MAPK) proliferation pathway, and phosphatidylinositol 3-kinase (PI3K)/protein kinase B (PKB or Akt) pro-survival pathway, that promotes tumor cell survival through progression of the cell cycle and inhibition of apoptosis.

HER2 is located at the long arm of human chromosome 17 (17q12). Amplification or overexpression of this oncogene has been shown to play an important role in the development and progression of certain aggressive types of breast cancer. In recent years, the protein has become an important biomarker and target of therapy for approximately 30% of breast cancer patients. As such, HER2 is the target of the monoclonal antibody trastuzumab, clinically approved and used for more than 20 years (marketed as Herceptin). Trastuzumab is effective only in cancers where HER2 is over-expressed. One year of trastuzumab therapy is recommended for all patients with HER2-positive breast cancer who are also receiving chemotherapy. An important downstream effect of trastuzumab binding to HER2 is an increase in p27, a protein that halts cell proliferation. Another monoclonal antibody, pertuzumab, which inhibits dimerization of HER2 and HER3 receptors, was approved by the FDA in 2012 for use in combination with trastuzumab.

Immunoglobulins or antibodies specifically bind antigens through the antigen binding site. The common immunoglobulin monomer is a “Y” shaped molecule that consists of four polypeptide chains; two identical heavy chains and two identical light chains connected by disulfide bonds. Variable loops of β-strands, three each on the light (VL) and heavy (VH) chains are responsible for binding to the antigen. These variable loops are the complementarity-determining regions (CDRs). Nanobodies (Nbs) are single-domain antibodies with unique biophysical properties, naturally occurring in the camelidae family (e.g., Camel, Llama, and Alpaca). Nanobodies are composed of a heavy chain only Abs (HCAbs)-antigen-binding fragment, solely composed of a single variable domain, referred to as VHH (variable domain of the heavy chain of HCAbs), sized 15 kDa. These antibodies typically have excellent stability and high affinity to their antigens and as encoded by single genes they can be easily cloned and produced.

Altunay et al. disclose HER2-directed antibodies, affibodies and nanobodies as drug-delivery vehicles in breast cancer with a specific focus on radioimmunotherapy and radioimmuno imaging (European Journal of Nuclear Medicine and Molecular Imaging (2021) 48:1371-1389).

Yan et al. disclose small molecular antibody, HER2-Nanobody that inhibits tumor proliferation in HER2-Positive breast cancer cells in vitro and in vivo (Front. Oncol., 12 May 2021, Vol 21, Article 669393).

Feng et al. disclose Evaluation of an ¹³¹I-labeled HER2-specific single domain antibody fragment for the radiopharmaceutical therapy of HER2-expressing cancers (Nature, Scientific reports, (2022) 12:3020).

There is an unmet need to provide additional and more effective, specific, safe and/or stable agents that alone, in combination with other agents, and/or as a carrier to antitumor drugs, can be used diagnostically and therapeutically in cancer involving HER2 expression.

### SUMMARY OF THE INVENTION

The present invention provides according to some embodiments, binding molecules, in particular single domain molecules such as nanobodies or VHH antibodies, that bind with high affinity and specificity to human epidermal growth factor receptor 2 (HER2). The present invention further provides conjugates comprising said binding molecules. Pharmaceutical compositions comprising the binding molecules and methods of treating cancer using the binding molecules or conjugates are also provided according to certain embodiments.

Advantageously, the binding molecules of the present invention are small molecules that were found to internalize into target cells. Moreover, the binding molecules described herein are capable of targeting HER2 without interfering with HER2/HER3 dimerization.

Thus, the binding molecules described herein are highly suitable for binding HER2 and for targeting cytotoxic drugs to cancer cells expressing HER2.

It is further disclosed that the nanobodies described herein do not compete with anti HER2 antibodies currently in clinical use, such as Trastuzumab (Herceptin®) and thus, may be used simultaneously.

It is now disclosed that a nanobody described herein, termed Nb46, showed high affinity to HER2, did not interfere with HER2-HER3 dimerization and phosphorylation and internalized to SkBr3 target cells.

Unexpectedly, NB46 had no effect on HER2 receptor recycling in cancer cells. This contrasts with Trastuzumab/Pertuzumab antibodies that reduce the recycling of HER2 receptors. The reduction of HER2 expression on breast cancer cells can lead to the development of acquired resistance to HER2-targeted therapies such as Trastuzumab and Pertuzumab. Accordingly, the antibodies described herein exhibit different mechanism of action and can overcome or prevent the development of resistance in HER2 positive breast cancer.

According to one aspect, the present invention provides an anti-HER2 binding molecule, or a fragment, derivative or analog thereof, the binding molecule comprising a set of three complementarity-determining region (CDR) sequences wherein the set is selected from the group consisting of:
i. a set derived from VHH termed Nb46 comprising the CDR sequences: GYFYYDHYYVA (SEQ ID NO: 2), INGRDSD (SEQ ID NO: 3) and AANPGEAFTVLPPRVFRN (SEQ ID NO: 4); and
ii. a set derived from VHH termed Nb38 comprising the CDR sequences: GFTRSMG (SEQ ID NO: 6), INNYNIGSG (SEQ ID NO: 7), and AASPLYLCDNSSWFAAGFAAGSHV (SEQ ID NO: 8).

According to some embodiments, the anti-HER2 binding molecule or a fragment thereof comprising an amino acid sequence at least about 90% identical to a sequence selected from the group consisting of SEQ ID Nos: 1 and 5.

According to some embodiments, the binding molecule is a single chain antibody. According to some embodiments, the binding molecule has a single binding domain. According to some embodiments, the binding molecule is a heavy chain single-domain (VHH) antibody or antibody fragment, an analog or derivative thereof having at least 90% sequence identity with said binding molecule or fragment sequence. According to some embodiments, the binding molecule is a heavy chain single-domain (VHH) antibody.

According to certain embodiments, the anti-HER2 binding molecule or a fragment thereof comprising an amino acid sequence at least about 90%, 92%, 94%, 96%, or 98% identical to a sequence selected from the group consisting of SEQ ID Nos: 1 and 5, said binding molecule comprises the three CDRs described herein.

According to some embodiments, the binding molecule is a camelid antibody. According to other embodiments, the binding molecule is a llama antibody.

The present invention provides, according to some embodiments, a camelid single heavy chain variable domain (VHH) antibody that specifically binds HER2, or an analog or derivative thereof having at least 90% sequence identity with said binding molecule.

According to some embodiments, the binding molecule comprises at least 90%, 91%, 92%, 93%, 94%, 95%, 96%, 97%, 98%, or 99% sequence identity with SEQ ID NO: 1. Each possibility represents a separate embodiment of the invention.

According to some embodiments, the binding molecule comprises the amino acid sequence of SEQ ID NO: 1. According to some embodiments, the binding molecule, denoted herein Nb46, consists of an amino acid set forth in SEQ ID NO: 1.

According to some embodiments, the binding molecule comprises at least 90%, 91%, 92%, 93%, 94%, 95%, 96%, 97%, 98%, or 99% sequence identity with SEQ ID NO: 5. Each possibility represents a separate embodiment of the invention.

According to some embodiments, the binding molecule comprises the amino acid sequence of SEQ ID NO: 5. According to some embodiments, the binding molecule, denoted herein Nb38, consists of an amino acid set forth in SEQ ID NO: 5.

Also included within the scope of the present invention a variant of the binding molecule or a fragment having at least 95% identity with said binding molecule.

According to some embodiments, the binding molecule has increased binding affinity to HER2. According to some embodiments, the binding molecule binds to the HER2 protein with an affinity of 10⁻⁹ M to 10⁻¹¹ M. According to certain embodiments, the binding molecule binds to the HER2 protein with an affinity of 0.5×10⁻⁸ M to 10⁻¹⁰ M.

According to some embodiments, the binding molecule binds to the HER2 protein with an affinity of at least 10⁻⁸ M. According to certain embodiments, the binding molecule binds to the HER2 protein with an affinity of at least 10⁻⁹ M. According to certain embodiments, the binding molecule binds to the HER2 protein with an affinity of about 0.8×10⁻⁹ M.

According to some embodiments, the binding molecule is characterized by molecular weight of less than 50 kDa, less than 40 kDa, less than 30 kDa, or less than 20 kDa. Each possibility represents a separate embodiment of the invention. According to some embodiments, the binding molecule is characterized by molecular weight of between about 14-16 kDa.

According to some embodiments, the binding of the binding molecule to HER2 is characterized by allowing further interaction(s) to HER2. According to some embodiments, the binding of the binding molecule to HER2 is characterized by retaining HER2 activity. According to some embodiments, the binding of the binding molecule to HER2 is characterized by retaining HER2 capability of dimerization with HER3. According to some embodiments, the binding of the binding molecule to HER2 receptor is characterized by allowing recycling of the HER2 receptor to the cell membrane following the internalization of the receptor-binding molecule complex. According to certain embodiments, the binding of the binding molecule to HER2 receptor is characterized by allowing recycling of the HER2 receptor to the cell membrane within 1-2 hours following the internalization of the receptor-binding molecule complex.

Analogs and derivatives of the binding molecules and the fragments described above, are also within the scope of the invention.

According to some embodiments, the binding molecule comprises a hypervariable region (HVR) comprising a set of three CDR sequences defined above, in which 1, 2, 3, 4, or 5 amino acids were substituted, deleted and/or added. Each possibility represents a separate embodiment of the invention.

According to some embodiments, the binding molecule or fragment comprises at least one CDR as defined above, in which 1, 2, or 3 amino acids were substituted. According to specific embodiments, the binding molecule or fragment comprises at least one CDR as defined above, in which one amino acid was substituted.

According to some embodiments, the binding molecule comprises at least one non-naturally occurring amino acid. According to some embodiments, the binding molecule comprises between 1-10, 1-5, 2-4, 3-5, 4-6, or 5-10 non-naturally occurring amino acid.

According to some embodiments, the CDR regions of the present invention are defined according to the Kabat method (Kabat Elvin A., Sequences of proteins of immunological interest, Bethesda, MD: U.S. Dept. of Health and Human Services, Public Health Service, National Institutes of Health, 1991), which is generally used in the single domain antibody field.

According to a specific embodiment, the fragment comprises at least the antigen-binding portion of the binding molecule.

The present invention provides according to another aspect, a construct, conjugate or fusion protein comprising at least one binding molecule specific to HER2 as described herein.

According to some embodiments, a conjugate comprising the anti-HER2 binding molecule or fragment thereof as described herein is provided.

According to some embodiments, the binding molecule is attached to a cytotoxic moiety, a radioactive moiety, an imaging agent, or an affinity or labeling tag.

According to some embodiments, the affinity tag is a peptide or an antibody. According to certain exemplary embodiments, the affinity tag is streptavidin.

According to some embodiments, the labeling tag is a functional group having a chemical reactivity.

According to some embodiments, the binding molecule is conjugated to toxin (payload). According to certain embodiments, the toxin is selected from the group consisting of microtubule inhibitor, DNA synthesis inhibitor, topoisomerase inhibitor and RNA polymerase inhibitor.

According to some embodiments, the toxin is directly connected to the binding molecule. According to other embodiments, the binding molecule and the toxin are connected through a linker. According to some embodiments, the toxin is covalently connected to the binding molecule directly or through a linker.

According to some embodiments, the linker is cleavable. According to additional embodiments, the linker is not cleavable. According to some embodiments, the linker is an enzymatic cleavable linker. According to certain embodiments, the linker is a pH-sensitive linker. According to some embodiments, the linker is a reducible linker (e.g., sulfo-SPDB).

According to some embodiment, a fusion protein comprising the binding molecule or fragment thereof, and a tag is provided. According to some embodiments, the binding molecule is fused to HA-tag. According to some embodiments, the binding molecule is fused to His-tag. According to additional embodiments, the binding molecule is fused to both HA-tag and His-Tag. According to some embodiments, the HA-tag and His-Tag comprises the amino acids sequence of SEQ ID NO: 9.

The invention further provides a polynucleotide sequence encoding a binding molecule that binds HER2 as described herein.

According to some embodiments, the polynucleotide sequences encode a molecule selected from the group consisting of: a binding molecule or fragment thereof as described herein, and a conjugate comprising said binding molecule or fragment. Each possibility represents a separate embodiment of the present invention.

According to some embodiments, the polynucleotide sequence encodes a VHH antibody according to any one of SEQ ID Nos: 1 or 5, or a variant thereof having at least 90% sequence identity. According to certain embodiments, the polynucleotide sequence encodes a VHH antibody according to any one of SEQ ID Nos: 1 or 5. According to some embodiments, the polynucleotide sequence further encodes SEQ ID Nos: 9.

According to yet some embodiments, the polynucleotide sequence according to the invention encodes a binding molecule or fragment or chain comprising: a set of three CDRs selected from the group consisting of:
i. a set derived from VHH termed Nb46 comprising the CDR sequences: GYFYYDHYYVA (SEQ ID NO: 2), INGRDSD (SEQ ID NO: 3) and AANPGEAFTVLPPRVFRN (SEQ ID NO:4); and
ii. a set derived from VHH termed Nb38 comprising the CDR sequences: GFTRSMG (SEQ ID NO: 6), INNYNIGSG (SEQ ID NO: 7), and AASPLYLCDNSSWFAAGFAAGSHV (SEQ ID NO: 8).

According to another aspect, the present invention provides a construct comprising a polynucleotide sequence encoding at least one binding molecule specific to HER2 as described herein. According to some embodiments, the nucleic acid construct is a plasmid.

In still another aspect, the present invention provides a cell capable of producing at least one binding molecule specific to HER2 as described herein.

According to another aspect, the present invention provides a pharmaceutical composition comprising the binding molecule or fragment described herein, or a fusion protein or a conjugate comprising the binding molecule, and a pharmaceutically acceptable excipient, carrier, or diluent.

According to some embodiments, the pharmaceutical composition is for use in treating cancer.

According to some embodiments, the pharmaceutical composition is for use in treating HER2+ associated disease or disorder. According to some embodiments, the pharmaceutical composition is for use in treating HER2+ tumors.

According to some embodiments, the pharmaceutical composition is for use in treating breast, gastric, ovarian or colorectal cancer. According to certain embodiments, the pharmaceutical composition is for use in treating breast cancer.

Any administration mode may be used to deliver the pharmaceutical compositions of the present invention to a subject in need thereof, including parenteral and enteral administration modes.

According to some embodiments, the pharmaceutical composition is formulated for injection or infusion. According to some embodiments, the pharmaceutical composition is formulated for intravenous administration. In certain embodiments, the pharmaceutical composition is formulated for intratumoral administration.

According to yet another aspect, the present invention provides a method of treating cancer comprising administering to a subject in need thereof, a therapeutically effective amount of at least one binding molecule or conjugate as described herein.

According to some embodiments, the cancer is HER2+ cancer.

In certain embodiments, the cancer is selected from the group consisting of breast cancer, prostate cancer, colorectal cancer, liver cancer, ovarian cancer, endometrial cancer, stomach cancer, thyroid cancer, carcinoid tumor, head and neck cancer, pancreatic cancer, testis cancer, urothelial cancer, cervical cancer, melanoma, lymphoma and lung cancer. Each possibility represents a separate embodiment of the invention.

According to some embodiments, the subject is human.

According to some embodiments, the method of treating cancer comprises administering or performing at least one additional anti-cancer therapy. According to certain embodiments, the additional anticancer therapy is surgery, chemotherapy, radiotherapy, a biological therapy, or immunotherapy.

According to some embodiments, the additional anti-cancer therapy is an antibody against HER2. According to certain embodiments, the antibody is Trastuzumab (Herceptin). According to additional embodiments, the antibody is Pertuzumab (Perjeta).

According to some embodiments, the administering of the anti-cancer therapy and the binding molecule described herein is carried out substantially simultaneously, concurrently, alternately, sequentially or successively. According to certain embodiments, the administering of the additional anti-cancer therapy is carried out before or after the administration of the binding molecule described herein. According to additional embodiments, the administering of the additional anti-cancer therapy and the binding molecule described herein is carried out simultaneously.

According to some embodiments, the additional anti-cancer agent is selected from the group consisting of: immune modulator, activated lymphocyte cell, kinase inhibitor and chemotherapeutic agent. According to other embodiments, the additional immune modulator is an antibody.

According to an additional aspect, the present invention provides a method of diagnosing or prognosing HER2+ cancer in a subject, the method comprises determining the expression level of HER2 in a biological sample of said subject using at least one binding molecule, fragment or conjugate as described herein.

According to an additional aspect, the present invention provides a method of determining or quantifying the expression of HER2, the method comprising contacting a biological sample with a binding molecule or a fragment thereof as described herein, and measuring the level of complex formation.

According to some embodiments, the method for detecting or quantifying the expression of HER2 comprises the steps of:
(i) incubating a sample with the binding molecule specific to HER2 as described herein or a fragment thereof comprising at least an antigen-binding portion; and
(ii) detecting the bound HER2 using a detectable probe.

According to some embodiments, the method further comprises the steps of:
(iii) comparing the amount of bound HER2 of step (ii) to a standard curve obtained from a reference sample containing a known amount of HER2; and
(iv) calculating the amount of the HER2 in the sample from the standard curve.

According to some particular embodiments, the sample is a body fluid or a solid tissue sample. In some embodiments, the method is performed in-vitro or ex-vivo.

A kit for measuring the expression of HER2 in biological sample is also provided comprising at least one binding molecule or fragment as described herein and means for measuring HER2 expression. In some embodiment, the kit further comprising instruction material directing the use of the kit.

It is to be understood that any combination of each of the aspects and the embodiments disclosed herein is explicitly encompassed within the disclosure of the present invention.

These and further aspects and features of the present invention will become apparent from the detailed description, examples and claims which follow.

### BRIEF DESCRIPTION OF THE DRAWINGS

FIGS. 1A-1B. Stable Dubca cells analysis by FACS and imaging. FIG. 1A—Expression of HER2 and HER3 on stable HER2/3-Dubca cells was assayed by FACS. Positive expression signal was seen for HER2 (Y-axis) and for HER3 (X-axis). FIG. 1B—Immunofluorescence live imaging of HER2/3-expressing cells. The Human receptors HER3 and HER2 co-localize on the cell surface of Dubca cells following lentiviral infection, as can be seen on the merge image. Scale bar indicated at the bottom represents 10 μm. The images were captured after 1 hour of incubation at 4° C. with the anti-HER2-FITC and anti-HER3-APC antibodies.

FIGS. 2A-2C. Camel immune response to HER2/3 cells. FIG. 2A—Pre-immune serum from the camel was used and incubated lysates of WT (uninfected) or Dubca cells expressing the human receptors HER2&3. From left to right, membranes blotted with Dubca WT, HER2/3 were incubated with anti-camelid VHH-HRP Ab. As expected, no antibodies were generated against the human receptors prior to immunization. FIG. 2B—Serum from week 4 post immunization was incubated with the lysates of stable Dubca cells co-expressing HER2 and HER3 and cells expressing each receptor exclusively. The upper bands (marked in circle) indicate that the immunization elicited polyclonal antibodies against the human antigens HER2 and HER3. These prominent signals did not appear in the Dubca WT samples. FIG. 2C—Membranes were incubated with antibodies specific to HER3 and HER2 to validate the bands.

FIGS. 3A-3D. Nanobodies library construction. FIG. 3A—Total RNA was extracted from the camel lymphocytes and loaded on a 1% agarose gel to verify its integrity. The presence of two distinct bands suggests high-quality RNA, and 28S/18S ribosomal RNA (rRNA) ratio of approximately 2:1 indicating of an intact purified RNA. FIG. 3B—Nested PCR was used to amplify the nanobody fragments. The first PCR products represent the antibody VH-CH1-CH2 and VHH-CH2 fragments. FIG. 3C—The second PCR amplified the VHH/nanobody. FIG. 3D—Clones from the Nb library were randomly selected for colony PCR to detect the percentage of clones with a plasmid containing a nanobody. Of the 30 colonies, 27 colonies were positive with an insert of 700 bp.

FIG. 4. Periplasmic extract ELISA screen results. Individual nanobody clones were randomly selected from the fifth cell-based panning round, and their binding was screened against HER2/3-expressing Dubca cells. Nb clones, whose binding level to HER2/3 represented by absorbance values (of OD₄₅₀) was more than two-fold higher than values for the negative control (nanobodies binding with Dubca WT), were considered as positive, and were selected for sequencing.

FIGS. 5A-5B. Amino acid sequences and purification of selected nanobodies. FIG. 5A—Nbs directed against HER2/3 are divided into 3 classes based on their sequences. Nb23 amino acid sequence represent 8 Nb clones and shows and early stop codon. Class two, defined as Nb46 represent 18 Nb clones, and class three, defined as Nb38 represent 2 Nb clones. Sequence alignment between these two classes showed mostly differences in their CDR domains (antigen-binding loops), underlined, and few differences in their FR domain. FIG. 5B—SDS-PAGE gel analysis of purified nanobodies following Ni-NTA purification. Protein's yield were 1 mg/L and 1.2 mg/L for N38 and Nb46, respectively.

FIGS. 6A-6D. Nb46 binding to HER2 cell-receptors by FACS analysis. FIGS. 6A-6B—HEK 293 cells expressing HER2, HER3 or both (HER2/3) receptors were incubated with various concentrations of Nb46 for 1 hour before reaction with fluorescent anti-VHH-488 antibody. The fluorescent signal was subtracted from that of cells incubated with the antibody alone and normalized relative to the highest signal (FIG. 6B), n=3. FIG. 6C—Affinity binding of NB46 with SkBr3 cells conducted with and without 1.8 mM Neuregulin-1 (NRG) following 1 hour incubation. Data was fitted with a sigmoidal curve in Excel (dashed lines), n=3. FIG. 6D—binding of fluorescent Nb46 labelled with Alexa Flour 647 to SkBr3 cells is comparable with that of the unlabeled Nb46, indicated by the fluorescence of secondary anti-VHH-488 antibody and Alexa Flour 647 fluorescence (for NB46-AF647, curves added to guide the eye).

FIG. 7. NB46-AF647 shows binding to SkBr3 cells membranes and subsequent internalization into cells cytoplasm by laser confocal microscopy. Cells were incubated with 1 nM of Nb46-AF647 and imaged after 1 h and 17 h. HER2 receptor stained by a-HER2 Ab—488 and nuclei stained by Hoechst. Scale bar=20 μm.

FIGS. 8A-8C. SkBr3 cells viability following incubation with NB46 for 24 (FIG. 8A), 48 (FIG. 8B) and 72 h (FIG. 8C), measured by CellTiter-Glo, with or without 2.5 nM NRG.

FIGS. 9A-9B. NB46 shows no effect on the phosphorylation of HER2 and HER3. Western blot analysis of SkBr3 cells incubated with Nb46 at various concentrations with and w/o 2.5 nM NRG (marked+/−), normalized signals (FIG. 9A) and representative image (FIG. 9B).

FIG. 10. Structural analysis of Nb46. Solved crystal structures of Nb46 with CDRs 1, 2, and 3 as indicated.

FIG. 11. HER2 recycling via immunofluorescence in SKBR3 cancer cells over time. Representative immunofluorescence micrographs show cell surface HER2 in non-permeabilized cells. Scale bar 16 μm.

FIG. 12. NB46 conjugated to DM1-SMCC showed by Nanodrop (FIG. 12A) and MALDI-TOF MS (FIG. 12B).

### DETAILED DESCRIPTION OF THE INVENTION

The present invention provides binding molecules specific to HER2, drug conjugates comprising the binding molecules, and pharmaceutical compositions comprising thereof.

According to one aspect, the present invention provides an anti-HER2 binding molecule or a fragment thereof comprising an amino acid sequence at least about 90% identical to a sequence selected from the group consisting of SEQ ID Nos: 1 and 5.

The HER2 gene or erb-b2 receptor tyrosine kinase 2 (also known as NEU; NGL; TKR1; CD340; HER-2; VSCN2; MLN 19; c-ERB2; c-ERB-2; HER-2/neu) encodes for the HER2 protein (or HER2/neu), a member of the Human epidermal growth factor receptors (HERs). An exemplary HER2 protein according to the invention is set forth in UniPort and GenBank symbols or accession numbers: Gene ID: 2064, or P04626.

According to some embodiments, the binding molecule is a VHH antibody (nanobody) or a fragment thereof.

The term “binding molecule” as used herein, refers to an antigen binding protein comprising at least a heavy chain variable region (VH) that binds to a target epitope.

The binding molecules described herein comprise at least 3 CDRs.

There are several methods known in the art for determining the CDR sequences of a given antibody molecule, but there is no standard unequivocal method. Determination of CDR sequences from antibody heavy and light chain variable regions can be made according to any method known in the art, including but not limited to the methods known as KABAT, Chothia and IMGT. A selected set of CDRs may include sequences identified by more than one method, namely, some CDR sequences may be determined using KABAT and some using IMGT, for example. According to some embodiments, the CDR sequences of the mAb variable regions are determined using the IMGT method. For example, such determination is made according to the Kabat (Wu T. T and Kabat E. A., J Exp Med, 1970; 132:211-50) and IMGT (Lefranc M-P, et al., Dev Comp Immunol, 2003, 27:55-77).

When the term “CDR having a sequence”, or a similar term is used, it includes options wherein the CDR comprises the specified sequences and also options wherein the CDR consists of the specified sequence.

The antigen specificity of the binding molecule described herein is based on the hyper variable region (HVR), namely the unique CDR sequences of the heavy chain that form the antigen-binding domain (ABD).

The present invention provides in some embodiments single-domain antibodies from camelid, called VHH that specifically bind to HER2 protein present on human cells. In some embodiments, the VHH are used as a targeting vehicle for cytotoxic agents.

According to some embodiments, the antibody is a single chain antibody. According to some embodiments, the antibody is a heavy chain single-domain (VHH) antibody or fragment, or an analog or derivative thereof having at least 90% sequence identity with said VHH antibody or fragment sequence.

The term “single-domain antibody” refers to an antibody fragment consisting of a single variable domain (VHH). Single-domain antibody is a smaller functional fragment of the antibody that also can bind a specific antigen. According to some embodiments, the single-domain antibody of the present invention comprises three complementary-determining regions (CDRs).

According to some embodiments, the VHH is a camelid antibody.

The terms “VHH”, “nanobody”, or “VHH nanobody”, are used herein interchangeably and refer to single heavy chain variable domain antibodies devoid of light chains. Preferably a VHH is an antibody of the type that can be found in Camelidae which are naturally devoid of light chains or a synthetic and non-immunized VHH which can be constructed accordingly.

The term “Camelidae”, as is used herein, includes reference to Llamas such as, for example, Lama glama, Lama vicugna (Vicugna vicugna) and Lama pacos (Vicugna pacos), and to Camelus species including, for example, Camelus dromedarius and Camelus bactrianus.

VHH antibodies are encoded by specific VHH V-genes, which are about 20 genes that can be divided into 3 groups, by VH genes (Deschacht et al. J Immunol 2010; 184:5696-5704) and the fusion of these V-genes to the various D- and J-genes. Whereas the D-genes can encode a very large number of sequences there are only 7 J-genes in the genomes of lamas (Achour et al. J Immunol 2008; 181:2001-2009).

The term ‘binding’ as used herein in the context of binding between a binding molecule, preferably a VHH, and an epitope on human HER2 protein, refers to the process of a non-covalent interaction between molecules. Preferably, said binding is specific. The terms ‘specific’ or ‘specificity’ or grammatical variations thereof refer to the number of different types of antigens or their epitopes to which a particular antibody such as a VHH can bind. The specificity of an antibody can be determined based on affinity. A specific antibody preferably has a binding affinity Kd for its epitope of less than 10⁻⁷ M, preferably less than 10⁻⁸ M, most preferable less than 10⁻⁹ M.

The term epitope or antigenic determinant refers to a part of an antigen that is recognized by an antibody. The term epitope includes linear epitopes and conformational epitopes. A conformational epitope is based on 3-D surface features and shape and/or tertiary structure of the antigen.

The term affinity refers to the strength of a binding reaction between a binding domain of an antibody and an epitope. It is the sum of the attractive and repulsive forces operating between the binding domain and the epitope. It should be noted that the affinity can be quantified using known methods such as, Surface Plasmon Resonance (SPR), and can be calculated using a dissociation constant, Kd, such that a lower Kd reflects higher affinity.

The term “antigenic determinant” or “epitope” as used herein refers to the region of an antigen molecule that specifically reacts with a particular binding molecule. Peptide sequences derived from an epitope can be used, alone or in conjunction with a carrier moiety, applying methods known in the art, to immunize animals and to produce additional polyclonal or monoclonal antibodies. Isolated peptides derived from an epitope may be used in diagnostic methods to detect binding molecules.

The term “fragments” comprise only a portion of the binding molecules as described herein, generally including an antigen binding site of the binding molecule and thus retaining the ability to bind antigen.

Various techniques have been developed for the production of binding molecule fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from antibody phage libraries. Alternatively, antibody fragments can be directly recovered from E. coli. Other techniques for the production of antibody fragments will be apparent to the skilled practitioner.

According to some embodiments, the binding molecule is a monoclonal antibody. The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

Sequence identity is the number of amino acids or nucleotides which match exactly between two different sequences. Sequence similarity permits conservative substitution of amino acids to be determined as identical amino acids.

The invention also provides conservative amino acid variants of the binding molecules according to the invention. Variants according to the invention also may be made such to conserve the overall molecular structure of the encoded proteins. Given the properties of the individual amino acids comprising the disclosed protein products, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved. The term “binding molecule analog” as used herein refers to a binding molecule derived from another binding molecule by one or more conservative amino acid substitutions.

The term “binding molecule variant” as used herein refers to any molecule comprising the binding molecule of the present invention. For example, fusion proteins in which the binding molecule or an antigen-binding-fragment thereof is linked to another chemical entity is considered a variant.

Analogs and variants of the binding molecule sequences are also within the scope of the present application. These include, but are not limited to, conservative and non-conservative substitution, insertion and deletion of amino acids within the sequence. Such modification and the resultant analog or variant are within the scope of the present invention as long as they confer, or even improve the binding to the antigen (i.e., HER2).

Conservative substitutions of amino acids as known to those skilled in the art are within the scope of the present invention. Conservative amino acid substitutions include replacement of one amino acid with another having the same type of functional group or side chain, e.g., aliphatic, aromatic, positively charged, negatively charged. These substitutions may enhance oral bioavailability, penetration, and targeting to specific cell populations, immunogenicity, and the like. One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. For example, according to one table known in the art, the following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

It should be emphasized that the variant chain sequences are determined by sequencing methods using specific primers. Different sequencing methods employed on the same sequence may result in slightly different sequences due to technical issues and different primers, particularly in the sequence terminals.

According to some embodiments, the binding molecule described herein comprises a non-naturally occurring amino acid. Methods for integrating a non-naturally occurring amino acid into a polypeptide are common and would be apparent to one of ordinary skill in the art.

According to some embodiments, the binding molecule is characterized by molecular weight of less than 15 kDa, less than 20 kDa, less than 25 kDa, less than 30 kDa, less than 35 kDa, less than 40 kDa, or less than 50 kDa, or any value and range therebetween. Each possibility represents a separate embodiment of the invention.

According to some embodiments, the binding molecule is characterized by thermal stability (Tm) of at least 60° C., at least 70° C., at least 90° C., or at least 95° C., or any value and range therebetween. Each possibility represents a separate embodiment of the invention.

As used herein, the term “thermal stability” refers to a substance resistance to irreversible change in its chemical or physical structure at an elevated temperature. According to some embodiments, Tₘ indicates the thermal energy that caused the denaturation/unfolding of a protein or a peptide.

According to some embodiments, the N- or C-terminus of the binding molecule comprises a tag motif. According to some embodiments, the tag motif comprises at least six amino acids. According to some embodiments, the tag is selected from the group consisting of HA-tag, poly(His) tag, chitin binding protein (CBP), maltose binding protein (MBP), Strep-tag, glutathione-S-transferase (GST), FLAG-tag, Spytag, C-tag, ALFA-tag, V5-tag, Myc-tag, Spot-tag, and T7-tag. According to some embodiments, the binding molecule comprises histidine (His)-tag. According to some embodiments, the binding molecule comprises human influenza hemagglutinin (HA)-tag.

### Antibody-Drug Conjugates

According to an aspect, the present invention provides a conjugate comprising the binding molecule as described herein fused to a therapeutic agent. According to some embodiments, the therapeutic agent is a toxin.

According to an aspect, the present invention provides a conjugate comprising the binding molecule as described herein fused to a toxin.

According to some embodiments, the toxin is a chemotherapeutic agent. According to some embodiments, the toxin is selected from the group consisting of microtubule inhibitor, DNA synthesis inhibitor, topoisomerase inhibitor, and RNA polymerase inhibitor. Each possibility represents a separate embodiment of the invention.

According to certain embodiments, the toxin is a microtubule-destroying drug. According to certain exemplary embodiments, the toxin is auristatin or a derivative thereof. According to certain embodiments, the auristatin derivative is monomethyl auristatin E (MMAE) or monomethyl auristatin F (MMAF).

According to some embodiments, the toxin is saponin.

According to some embodiments, the toxin is a maytansine derivative. According to certain embodiments, the maytansine derivative is DM4 or DM1.

According to some embodiments, the toxin is quinoline alkaloid. According to certain embodiments, the quinoline alkaloid is SN-38.

According to additional embodiments, the toxin is selected from the group consisting of MMAE, MMAF, Saporin, DM4, DM1, SN-38, Calicheamicin, DXd, PBD, Duocarmycin, Sandramycin, alpha-Amanitin, Chaetocin, Daunorubicin, 17-AAG, Agrochelin A, Doxorubicin, Methotrexate, Colchicine, Cordycepin, Hygrolidin, Herboxidiene, Ferulenol, Curvulin, Englerin A, Taltobulin, Triptolide, Cryptophycin, and Nemorubicin.

According to some embodiments, the binding molecule is directly connected to the toxin. According to other embodiments, the binding molecule and the toxin are connected through a linker. According to some embodiments, the binding molecule described herein is covalently linked to the toxin.

According to some embodiments, the linker is cleavable. According to additional embodiments, the linker is not cleavable.

According to some embodiments, the linker is cleaved in response to changes in pH or redox potential. According to some embodiments, the linker is cleaved when contacted with lysosomal enzymes.

### Pharmaceutical Compositions

According to an aspect, the present invention provides a pharmaceutical composition comprising the binding molecule as described herein and, optionally at least one excipient, diluent, salt or carrier.

In pharmaceutical and medicament formulations, the active agent is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired exposure.

Typically, the binding molecules, fragments and conjugates thereof of the present invention comprising the antigen binding portion will be suspended in a sterile saline solution for therapeutic uses. The pharmaceutical compositions may alternatively be formulated to control release of active ingredient or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention, i.e., of a binding molecule or fragment, from such a matrix depends upon the molecular weight of the molecule, the amount of the molecule within the matrix, and the size of dispersed particles.

The pharmaceutical composition of this invention may be administered by any suitable means, such as intravenously, orally, topically, intranasally, subcutaneously, intramuscularly, intra-arterially, intraarticulary, intralesionally, intratumorally or parenterally. Ordinarily, intravenous (i.v.) administration is used for delivering antibodies.

### Methods of Treatment

According to yet another aspect, the present invention provides a method of treating cancer comprising administering to a subject in need thereof, a therapeutically effective amount of at least one binding molecule or conjugate as described herein.

According to yet another aspect, the present invention provides a method of targeting HER2 by contacting cells comprising HER2 with the binding molecule described herein, thereby targeting HER2.

As used herein the term “individual,” “patient,” or “subject” refers to individuals diagnosed with, suspected of being afflicted with, or at-risk of developing at least one disease for which the described compositions and method are useful for treating. According to some embodiments the individual is a mammal. According to some embodiments, the mammal is a mouse, rat, rabbit, dog, cat, horse, cow, sheep, pig, goat, llama, alpaca, or yak. According to some embodiments, the individual is a human.

The term “treatment” as used herein refers to both therapeutic treatment and prophylactic or preventative measures. Those in need of treatment include those already with the disorder as well to prevent recurrence.

As used herein the term an “effective amount” refers to the amount of a therapeutic that causes a biological effect when administered to a mammal. Biological effects include, but are not limited to, reduced tumor growth, reduced tumor metastasis, or prolonged survival of an animal bearing a tumor. A “therapeutic amount” is the concertation of a drug calculated to exert a therapeutic effect. A therapeutic amount encompasses the range of dosages capable of inducing a therapeutic response in a population of individuals. The mammal can be a human individual. The human individual can be afflicted with or suspected or being afflicted with a tumor.

The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

According to some embodiments, the method of treating cancer comprises administering the pharmaceutical composition as part of a treatment regimen comprising administration of at least one additional anti-cancer agent.

According to some embodiments, the method of treating cancer comprises administration of the binding molecule described herein and an additional anti-cancer agent. According to some embodiments, the additional anti-cancer agent is selected from the group consisting of: immune modulator, activated lymphocyte cell, kinase inhibitor and chemotherapeutic agent.

According to some embodiments, the immune modulator is an antibody against an immune checkpoint molecule. According to some embodiments, the immune modulator is an antibody against an immune checkpoint molecule selected from the group consisting of human programmed cell death protein 1 (PD-1), PD-L1 and PD-L2, carcinoembryonic antigen-related cell adhesion molecule 1 (CEACAM1), lymphocyte activation gene 3 (LAG3), CD137, OX40 (also referred to as CD134), killer cell immunoglobulin-like receptors (KIR), TIGIT, PVR, CTLA-4, NKG2A, GITR, and any other checkpoint molecule or a combination thereof. Each possibility represents a separate embodiment of the invention. According to certain embodiments, the immune modulator is an antibody against PD-1. According to some embodiments, the additional immune modulator is an antibody against CTLA-4.

According to some embodiments, the anti-cancer agent is selected from the group consisting of: erbitux, cytarabine, fludarabine, fluorouracil, mercaptopurine, methotrexate, thioguanine, gemcitabine, vincristine, vinblastine, vinorelbine, carmustine, lomustine, chlorambucil, cyclophosphamide, cisplatin, carboplatin, ifosfamide, mechlorethamine, melphalan, thiotepa, dacarbazine, bleomycin, dactinomycin, daunorubicin, doxorubicin, idarubicin, mitomycin, mitoxantrone, plicamycin, etoposide, teniposide and any combination thereof. Each possibility represents a separate embodiment of the invention.

According to some embodiments, the method of treating cancer involves preventing or reducing formation, growth or spread of metastases in a subject.

According to another aspect, the present invention provides a method for imaging HER2 comprised cells in a subject, the method comprising administering to the subject a composition comprising the binding molecule described herein, and an imaging agent.

According to some embodiments, the imaging agent is selected from the group consisting of fluorescent label, a chromophore, a radioactive label, a paramagnetic ion, and any combination thereof.

According to some embodiments, the imaging agent is a radioactive label (e.g., isotope). Suitable radioactive labels include, for example, ¹⁸F, ⁴⁷SC, ⁵¹Cr, ⁵²Fe, ^{52m}Mn, ⁵⁶Ni, ⁵⁷Ni, ⁶²Cu, \hat{ }Cu, ⁶⁷Ga, ⁶⁸Ga, ⁷²As, ⁷⁵Br, ⁷⁶Br, ⁷⁷Br, ⁸²Br, ⁸⁹Zr, ^{94m}Tc, ⁹⁷Ru, ^{99m}Tc, ^{U1}In, ¹²³I, ¹²⁴I, ¹³¹I, ¹⁹¹Pt, ¹⁹⁷Hg, ²⁰¹Tl, ²⁰³Pb, ^{110m}In, ¹²⁰I, UC, ¹⁸F, and ¹³N.

According to some embodiments, the binding molecule is directly connected to the imaging agent. According to other embodiments, the binding molecule and the imaging agent are connected through a linker. According to some embodiments, the binding molecule described herein is covalently linked to the imaging agent.

### Methods of Manufacturing the Binding Molecules

The binding molecules of the invention can be produced by a variety of methods know in the art.

The binding molecules of the present invention may be produced by any method known in the art, including but not limited to transgenic production in microorganisms such as E. coli, Saccharomyces cerevisiae and Pichia pastoris, production in non-human mammalian cells, and production in plants.

According to some embodiments, the VHH antibodies of the present invention are produced in transgenic plants. According to additional embodiments, the VHH antibodies of the present invention are produced in genetically modified microorganisms, for example in bacteria or yeast, using methods well known in the art for producing recombinant proteins. According to other embodiments, the VHH antibodies are produced by chemical synthesis, using peptide synthesis methods well known in the art, for example solid-phase or liquid-phase synthesis.

The heavy chain variable domains may be derived in any suitable manner and from any suitable source, for example naturally occurring VHH sequences (i.e. from a suitable species of Camelid) or synthetic or semi-synthetic heavy chain variable domains, including but not limited to “camelized” immunoglobulin sequences, as well as those that have been obtained by techniques such as CDR grafting, combining fragments derived from different immunoglobulin sequences, PCR assembly using overlapping primers, and similar techniques for engineering immunoglobulin sequences well known to the skilled person.

In one exemplary embodiment, the antibodies may be produced by immunizing an animal with HER2. In some embodiments, the animal is a camel or llama. In other embodiments, the antibodies can be recombinantly produced in any expression know in the art, for example, yeast.

In some embodiments, suitable binding molecules are screened and identified using the phage display system.

Phage-display allows for targeted selection of antigen specific antibodies from antibody libraries and has been successfully applied to isolate antibodies specific for toxins of different origins (see e.g., Kuhn et al., 2016. Proteomics Clin Appl 10: 922-948). As demonstrated in the present invention, Camelid VHH libraries (the variable domains of the heavy-chains of heavy-chain only antibodies from camelids), were generated by immunization with cells presenting HER2. The phage display system was used for screening an identifying VHH antibodies that bind to HER2.

As used herein the term “about” refers to an amount that is near the stated amount by 10% or less.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological, immunological and recombinant DNA techniques. Such techniques are well known in the art. Other general references referring to well-known procedures are provided throughout this document for the convenience of the reader.

### Materials and Methods

Lentiviral production in HEK293T cells—Vectors containing coding sequences of Human HER2 (MSCV-human Erbb2-IRES-GFP) and HER3 (pDONR223-ERBB3) were obtained from Addgene (Cambridge, MA, USA), and were re-cloned into pHAGE2 lentiviral backbone vector (kindly provided by the Ran Taube lab, Ben-Gurion University, Israel) under the control of a CMV promoter, and with a puromycin resistance selection marker. The mRNA positions used for cloning listed in Table 1. The adherent human embryonic kidney HEK293T cells (ATCC, VA, USA) were transfected with each gene of interest using the packaging HIV expression plasmids pGag-Pol, pRT, pTat and pREv and the envelope VSV-G protein (kindly provided by the Ran Taube lab, Ben-Gurion University, Israel) by the transfection reagent polyethylenimine (PEI) (Polysciences, Inc. PA, USA), at a DNA:PEI weight ratio of 1:3 (g:g). Cells supernatant containing the lentivirus was collected 48 hours post-transfection, cleared through a 0.45 μm filter, aliquoted and stored at −80° C. for further use.

TABLE 1
mRNA positions used for cloning. The coding sequences
translated to the full-length proteins, are presented.

| | NCBI Reference | Coding sequence |
|---|---|---|
| Gene Name | Sequence (mRNA) | positions (mRNA) |
| HER2/ERBB2 | NM_004448.3 | 262-4029 |
| HER3/ERBB3 | NM_001982.4 | 137-4165 |

Generation of antigen-expressing cells—The adherent Dubca (Dubai camel) fibroblast cell line (ATCC, VA, USA) were seeded at 8×10⁴ cells/well in 12-well plates and infected with HER2- or HER3-lentivirus. Control Dubca cells were infected with empty_GFP-pHAGE2-lentivirus as a control for infection efficiency of the cells. For efficient infection, the previously described spin-fection protocol was used (Kodaka et al. Biotechniques. 2017; 63:72-76). Briefly, the lentivirus supernatant was added to cells in the 12-well plates with 10 μg/ml Polybrene (Sigma-Aldrich, Rehovot, Israel) per well, followed by centrifugation at 800×g for 90 min, at 37° C. Plates were then placed in a humidified incubator at 37° C./5% CO₂, and after 3-6 hours the medium was replaced with a fresh EMEM medium (ATCC, VA, USA) supplemented with 10% fetal bovine serum (FBS), 100 U/ml penicillin, 100 mg/ml streptomycin (Biological Industries, Beit Haemek, Israel). After 48 hours, cells were subjected to selection using Puromycin antibiotic (Sigma-Aldrich, Rehovot, Israel) at 2.5 μg/ml per well. To generate cells that express both HER2 and HER3, Dubca cells expressing HER3 were infected for a second time with HER2-lentiviruses, and then subjected to a double-selection sort by the FACS (BD FACS Aria II, Ilse Katz Institute for Nanoscale Science & Technology, BGU) using human HER2 Fluorescein isothiocyanate (FITC)-conjugated Ab (Biolegend, CA, USA) and human HER3 Allophycocyanin (APC)-conjugated Ab (R&D systems, MN, USA). These Abs were also used to detect co-localization of HER2 & HER3 on Dubca cells by the laser-scanning microscope ZEISS LSM-880 confocal microscope (Ilse Katz Institute for Nanoscale Science and Technology Shared Resource Facility, BGU) prior to sort. For a control sample, cells were incubated with the secondary Abs to determine the fluorescence background signals.

Immunization of a camel with HER2/3 expressing Dubca cells—To induce a humoral immune response directed towards the human cell surface proteins (HER2/HER3, PAR1), a 3 year old female camel (Camelus dromedaries) was immunized subcutaneously (above the shoulder at the beginning of the neck) eight times with one-week intervals, with approximately 1×10⁸ of a mixture of Dubca cells expressing HER2/3- and PAR1- (Cells infected to express PAR1 transmembrane protein). In order to maintain the open conformation of HER3, prior to the cells' preparation for injection, HER2/HER3-expressing cells were treated with 50 ng/ml Neuregulin (NRG1, the natural ligand for HER3, Biolegend, CA, USA) and incubated for 15 min at 37° C. Cells were washed with warm phosphate buffered saline (PBS), followed by cells dissociation with non-enzymatic cell dissociation solution (Biological Industries, Beit Haemek, Israel). After centrifugation at 150 g for 5 min and supernatant removal, the cells were gently fixed with 0.2% fresh paraformaldehyde (PFA) (Sigma-Aldrich, Rehovot, Israel) mixes in PBS (v/v) for 10 min at room temperature (RT), to preserve the conformation of HER3 in the complex HER2/3. Cells were again centrifuged and washed three times with PBS, and resuspended with 1 ml PBS, inserted into a sterile 5 ml syringes and kept on ice until injection to the camel. Right before the injection step, the cells were gently mixed with Gerbu P adjuvant (Gerbu Biotechnic, Gaiberg, Germany), at a ratio of 1:1 (vol/vol) in a final volume of 2-3 ml. To eliminate the risk of the adjuvant interrupting the receptors or compromising their conformational epitopes, for the 4^{th} and the 6^{th} immunizations the cells were not mixed with adjuvant, rather, cells were injected subcutaneously and separately at 5 cm distance, an equal volume of Gerbu P was injected to locally boost the immune system. All camel experiments were performed according to guidelines approved by the Israel Ethic Committee.

Immune monitoring—The immune response of the camel was monitored by collecting a small blood samples at pre-immune step and after four weeks post injection, and testing the serum by western blot as follows; Dubca WT (uninfected), and HER2/3 expressing cells were lysed by cold RIPA buffer (Sigma-Aldrich, Rehovot, Israel), and loaded on SDS-PAGE 10% gel, blotted onto a nitrocellulose membrane (Bio-rad, CA, USA), and incubated with 40 μl of the camel serum diluted in 10 mL 5% Bovine serum albumin (BSA) in TBST buffer, followed by a secondary anti-camel-VHH-HRP Ab (1:5000, ABclonal, MA, USA). The chemiluminescent signal was developed using EZ-ECL Kit (Biological Industries, Beit Haemek, Israel), and visualized by the Fusion FX imager (Vilber Lourmat, Eberhardzell, Germany). To validate that the detected bands on the membrane represent the HER2 and HER3 antigens the membranes were also incubated with mouse anti-human HER3 Ab (R&D systems, MN, USA) or with mouse anti-human HER2 Ab (GenomMe, Richmond, Canada), followed by incubation with a secondary HRP-anti-mouse IgG (Sigma-Aldrich, Rehovot, Israel).

Construction of VHH libraries—Four days after the final 8^{th} immunization, 100 ml of peripheral blood was collected, and blood lymphocytes (PBLs) were purified by density gradient centrifugation using Ficoll-Paque PLUS gradient media (GE Healthcare, WI, USA) according to the manufacturer's protocol. Total RNA was extracted from PBLs by TRIzol (Invitrogen, CA, USA) and transcribed into cDNA using Verso cDNA Synthesis Kit (Thermo Fisher Scientific, MA, USA). The cDNA pool was used as template for amplifying the Nbs library, in two steps of nested PCR; the first amplification generated two distinct groups of PCR amplicons with molecular weights of about 0.7 and 0.9 kb, with primers CALL001 and CALL002. For the second PCR, the 0.7 kb PCR amplicon was used as a template to amplify a 0.4 kb Nb fragment, with primers PMCF and VHH-BACK (Table 2). This nested PCR also introduces restriction enzyme sites of PstI at the 5′ end and NotI at the 3′ end of the Nbs amplicons for cloning into the phage display pMECS vector (containing C-terminal haemagglutinin, HA and hexahistidine, 6×His tags, kindly gifted by Dr. Serge Muyldermans, Vrije University Brussels, Brussels, Belgium). The pool of amplified Nb DNA in the phage-display vector pMECS was transformed into E. coli TG1 electrocompetent cells (Lucigen, WI, USA) to generate a library of transformants, as described earlier (Marsh et al. Springer, 2018. p 71-94).

TABLE 2
Primers used for PCR amplification

| Primer name | SEQ ID NO: | Sequence |
|---|---|---|
| CALL001 | 10 | GTCCTGGCTGCTCTTCTACAAGG |
| CALL002 | 11 | GGTACGTGCTGTTGAACTGTTCC |
| PMCF | 12 | CTAGTGCGGCCGCTGAGGAGACGGTGA |
| | | CCTGGGT |
| VHH-BACK | 13 | GATGTGCAGCTGCAGGAGTCTGGAGGA |
| | | GG |

Cell-based panning with Phage-display—Nb-displaying bacteriophage library was prepared as previously described following an M13K07 phage (New England BioLabs, MA, USA) infection according to the manufacturer's protocol. Briefly, the Nb library in TG1 cells was infected with 1.0×10¹¹ M13K07 helper phages to create a phage library. Filamentous phage is a flexible cylindrical shaped virus particle coated with five different coat proteins (pIII, pVIII, pVI, pVII and pIX), and the phage pIII is able to display the Nb variants on its surface. The cell-based panning rounds were designed to optimize a method for screening large number of clones in a simple and systematic fashion. The in vitro panning phage-display was performed against viable, adherent cell cultures in a total of 5 subsequent rounds starting with a negative screen followed by a positive screen, as follows: after phage production, 2×10¹¹ Nb-displaying phages were incubated with uninfected WT Dubca cells in T25 flasks, for a negative screen, at 30° C. for 1 h, 50 rpm. After the incubation, the supernatant of unbound phages from one flask was transferred to a flask containing the HER2/3-overexpressing Dubca cells, for a positive screen, at 30° C. for 1 h, 50 rpm. Prior to each panning, 4×10⁵ cells were seeded on a T25 flask with 5 ml complete EMEM medium and allowed to adhere and proliferate for 48 h in a humidified incubator at 37° C. and 5% CO₂. After the positive screen, the supernatant was removed and cells were washed 3 times with PBS supplemented with 5% FBS (v/v) at 37° C., for 5 min, 50 rpm. Elution of bound phages was done in acidic conditions by adding 2 ml of Glycine-HCl/NaCl pH 2.2 (100 mM glycine, 0.5 M NaCl, mixed in ddH₂O. After dissolving, pH was adjusted with 37% HCl to 2.2), as previously describes, to cover the cells in the flask, for 15 min. The eluted phages were centrifuged at 14,000 rpm, for 5 min. The supernatant was transferred into a new tube and the acidic buffer was neutralized by adding 50 μl of 2 M unbuffered Tris-base (24.22 gr Tris powder mixed in 100 ml ddH₂O, pH ˜10.5). At the end of a panning round, Nb-displaying phages were transferred into a naive TG1 E. coli culture and reamplified for further use in the next panning round, in the following day. Lastly, ˜10 μl of eluted phages were incubated with 100 μl of naive TG1 E. coli culture and spread on LB-ampicillin glucose plates (25 gr of LB medium and 15 gr of agar mixed in 900 ml of ddH₂O, supplemented with 100 ml of 20% (wt/vol) glucose and 1 ml of ampicillin (Amp) 100 mg/ml) and incubated at 37° C. overnight. These plates represent the clones of the library population after each panning round. These colonies contain single Nb clones that were used for the next Periplasmic enzyme-linked immunosorbent assay (ELISA).

Selection of high-affinity Nb binders—After five rounds of panning against HER2/3, Nbs-containing colonies were individually evaluated for HER2/3 binding using Periplasmic-ELISA, as described in Marsh et al. (Springer, 2018. p 71-94.) with modification. To prepare E. coli TG1 periplasmic extracts containing Nb, the colonies were prepared as follows; 188 colonies were randomly picked from the LB-ampicillin glucose plates and grown in 24-deep well plates (Sigma-Aldrich, Rehovot, Israel) with 2 ml TB medium (12 gr of Bacto tryptone, 2.3 gr of KH₂PO₄, 12.5 gr of K₂HPO₄, 24 gr of yeast extract and 2.5 ml of glycerol mixed in 1 liter of ddH₂O) containing Amp (100 g/ml), at 37° C. for 6 h, while shaking at 150 rpm. In order to induce the production of each Nbs and export to the periplasm space, 10 μl of 100 mM isopropyl-beta-D-thiogalactopyranoside (IPTG, Thermo Fisher Scientific, MA, USA) was added to each well, and incubated at 28° C. overnight, while shaking at 200 rpm. Each colony was also streaked on a new LB-ampicillin glucose plates for backup. The next day, the culture was centrifuged at 3,200 g for 10 min, and the pellet was freeze-thawed by incubation at −20° C. for 3 hours followed by pellet thaw at RT for 15 min. To release the Nbs from the TG1 periplasm, 400 μl of PBS was added to each well and the plate was incubated at RT for 30 min on a vibrating platform (700 rpm). Finally, plates were centrifuged at 3,200 g, 4° C., for 10 min to pellet the cell debris and recover the Nbs in the supernatant. Periplasmic Nbs from each colony were tested for antigen recognition, and 100 μl of each Nb was incubated with either HER2/3-overexpressing Dubca cells in a 96-well plate or uninfected WT cells, which served as control, at 30° C. for 1 h, with 50 rpm. Cells were washed 3 times with PBS supplemented with 5% FBS, and detection Nb binding was performed by using a mouse anti-HA antibody (1:2000, Biolegend, CA, USA), and secondary HRP-anti-mouse IgG (Sigma-Aldrich, Rehovot, Israel), followed by adding 100 μl TMB-ELISA Substrate Solution (Thermo Fisher Scientific, MA, USA) to each well. The reaction was stopped after ˜10 min by adding 100 μl of 1 M sulphuric acid (Sigma-Aldrich, Rehovot, Israel), and the absorbance of optical density (OD₄₅₀ nm) was read by a microplate Reader M1000 (Tecan Grödig, Austria), and the signal was reduced form the OD₄₅₀ nm of the control of each sample. For sequence determination, the selected Nbs with the highest absorbance values, were further amplified by PCR and sequenced with primers MP57 (5′-TTATGCTTCCGGCTCGTATG-3′; SEQ ID NO: 14) and GIII (5′-CCACAGACAGCCCTCATAG-3′; SEQ ID NO: 15) at the NIBN Sequencing unit, Ben-Gurion University of the Negev, Israel.

Recombinant expression and purification of nanobodies—Selected Nbs (in pMECS plasmid) were transformed into WK6 E. coli via heat-shock and secreted into the periplasm after an overnight induction with 1 mM IPTG at 28° C., while shaking at 250 rpm. Periplasmic extracts containing the soluble Nb's were obtained by osmotic shock using 12 ml of TES buffer (24.22 gr of Tris, 0.19 g of EDTA and 171.15 g of sucrose mixed in 1 liter of ddH₂O, pH 8.0) at 4° C. for 3 h, on an orbital shaking platform, followed by incubation with 24 ml of TES/4 buffer (diluted 1:4 with ddH₂O) at 4° C., overnight, with agitation. The next morning, the suspension was centrifuged at 10,000 g for 30 min, 4° C., and the supernatant was recovered. The His-tagged Nb from the supernatant were further purified using immobilized metal ion affinity chromatography by Nickel-NTA (Ni-NTA) beads (Invitrogen, CA, USA) in a gravity column, according to the manufacturer's instructions. After loading the supernatant, the nickel beads were washed with 20 column volumes of PBS supplemented with 10 mM Imidazole (Acros Organics, Geel, Belgium), followed by a secondary wash by PBS supplemented with 0.1% Triton X-114 (Sigma-Aldrich, Rehovot, Israel) to further remove E. coli endotoxins. Nbs were eluted with 15 ml PBS supplemented with 500 mM Imidazole, followed by dialysis against PBS overnight, and concentration using a Vivaspin with a cutoff of 5-kDa (Vivaproducts, MA, USA). The yield of each purified protein was determined using a NanoDrop spectrophotometer (Denovix, DE, USA), and purity and size was confirmed by SDS-PAGE gel stained with Coomassie Brilliant Blue (Bio-rad, CA, USA).

SPR binding assay—The affinity of Nbs to HER2 or HER3 was determined in two ways by using surface plasmon resonance (SPR) spectroscopy. SPR measurements were performed on a Biacore T200 instrument. In one layout, purified Nb38 (10 μg/ml) and Nb46 (20 μg/ml) were immobilized on a CM5 chip using an amine coupling kit (GE Healthcare, Chicago, IL). The analytes, human HER2-His or HER3-His extracellular-domain proteins (R&D systems, Minneapolis, MN) dissolved in PBST (PBS supplemented with 0.05% Tween-20 v/v), were flown at 30 μl/min, and the chip was regenerated using 2 mM NaOH. In a second layout, the purified human HER2-His or HER3-His extracellular-domain proteins, at 10 μg/ml, were immobilized on a CM5 chip). The analytes, NB46 and NB38 (and NB7 as a negative control), were flown at 30 μl/min in PBST, and the chip was regenerated using 20 mM glycine. Binding was allowed for 180 s and dissociation for 600 s. Curves were fitted using Biacore's evaluation software using a 1:1 ligand:analyte binding model to retrieve association rate constants (ka), dissociation rate constants (kd), and equilibrium dissociation constants (K_{D}, a measurement of affinity).

Nb thermal stability—The protein unfolding temperature (Tm) was inferred based on differential scanning fluorimetry (DSF) measurements performed on Prometheus NT.48 (NanoTemper, München, Germany). Samples of 0.3 mg/mL Nb46 in PBS were heated from 20° C. to 95° C. at 1° C./min. Tryptophan and tyrosine fluorescence was monitored by recording the 350/330 nm emission ratio after excitation at 280 nm and the Tm was taken at the ratio's inflection point.

HER2+ cancer cells viability—SkBr3 (7,000 cells) were seeded in wells of a 96 wells plate and incubated in complete RMPMI medium for 24 h. Thereafter, the medium was aspirated and replaced with fresh medium containing 50-5000 nM Nb-46, either supplemented with 2.5 nM NRG or without. Cells containing fresh medium and ones supplemented with only NRG were used as controls. Following overnight incubation, CellTiter-Glo© Luminescent reagent (Promega, Madison, WI) was added to each well in same volume as the medium, 100 μL and the plate was placed on an orbital shaker for 10 minutes. The wells contents were transferred into an opaque-walled 96 wells plate and plate was read for luminescence using a 528/20 nm filter on a Synergy2 microplate 7 spectrophotometer (BioTek, Winooski, VT), n=3. All signals were divided by that for cells with no Nb (for +NRG and −NRG separately).

Nb46 binding of HER2+ cancer cells—SkBr3 cells were grown to 70-90% confluency in T-75 flasks overnight. The cells were detached by aspirating the medium and washing cells with 10 mL hot PBS before pipetting 5 mL of non-enzymatic cell dissociation solution (Biological Industries, Beit Haemek, Israel) and incubating for 5 minutes at 37° C. The cells were added with 10 mL of PBS containing 2.5% FBS (PBS-FBS), transferred into a 50 mL tube and centrifuged for 5 minutes at 150 g before being resuspended in 1 mL of PBS-FBS counted and transferred (10 uL, 5·10⁴ cells) into separate Eppendorf tubes containing Nb46 or Nb46-AF647 at various concentrations 25-0.012 nM and volumes 300 uL-13 mL, compatible to avoid ligand depletion. The cells were Incubated for 1 h at 4° C. on a tubes rotator after which the cells were washed off of unbound Nb; cells were centrifuged at 350 g for 5 minutes, supernatant discarded, and cells resuspend in 900 μL of PBS-FBS, repeatedly for 3 times. After the 3^{rd} centrifugation, the cells were resuspended in 300 μL PBS-FBS containing iFluor488-conjugated anti-camelid VHH (1:1000, Genescript, NJ, USA), and incubated for additional 1 h at 4° C. on a tubes rotator, in the dark. Cells were thereafter washed as aforementioned, for 3 times and resuspended in 500 μL PBS-FBS directly before analysis on BD FACSCanto II (BD Biosciences, Erembodegem, Belgium). Cells stained with the 2^{nd} Ab only were served as control.

Nb46 binding and internalization into HER2+ cancer cells by live imaging—SkBr3 cells, 3×10⁴, were grown overnight in an 8-well μ-slide (ibidi GmbH, Germany) before adding 488-anti HER2 antibody (BioLegend, CA) and Hoechst 33342 (Invitrogen) and 1 nM N46-Af647 (produced from reaction of Nb46 with Alexa Fluor 647 NHS ester (Thermo Scientific, IL)), and live-imaged for up to 18 hours on a 3i Marianas (Denver, CO) spinning disk confocal microscope equipped with Yokogawa W1 module and Prime 95B sCMOS camera. Cells unreacted with Nb46-AF647 were served as control.

HER2 and HER3 phosphorylation—SKBR3 Cells were seeded in a 6-wells plate (300,000/well) and cultured for 24 hours in RPMI complete medium (+10% FBS, 1% P/s, 200 μL). The next day, the medium was replaced same volume of starvation medium (RPMI+1% p/s, 0.2% FBS), and cells were incubated for overnight at 37° C. On the following day, Nb46 was added in various concentrations, 1-5000 nM by dilution of a stock solution (110 PM), alongside cells with no added Nb, and cells were incubated for 10 minutes at 37° C. NRG1-01 (Biolegend, CA, USA) was then added to by dilution of the stock solution (34.2 μM, 0.95 mg/mL), to a final concentrations of 2.5 nM, alongside un-supplement cells and cells were further incubated for 15 minutes at 37° C. Cells were washed twice with ice cold PBS supplemented with 1 mM Na₃VO₄ (200 μL) and were then topped with 150 μL of lysis buffer (2% NP-40, 20 mM Tris (pH 8.0), 137 mM NaCl, 10% glycerol, 2 mM EDTA, 1 mM sodium orthovanadate (Na₃VO₄) mixed with protease inhibitor, APE×BIO by dilution 100:1 and incubated on ice for 10 minutes for the cells lysis. Cells were then scraped using a cell scraper, transferred to a 1.5 ml tubes and moved to the freezer (−20° C.) until the next day. The thawed lysed cells were centrifuged at 14,000 g at 4° C. for 15 minutes, the supernatant was collected from each tube to a mini-Eppendorf tubes and the Protein concentration of each sample was assessed by using the BCA kit, following the manufacturer's protocol (Pierce). Samples concentrations were equalized by diluting the concentrated samples to match their concentrations with that of the less concentrated sample and each sample was loaded on a 10%, 1.5 mm thick polyacrylamide gel (˜40 μg). PAGE was run at 120 V for approximately an hour. For western blot, the protein bands were transferred onto nitrocellulose membranes (0.45 μm), followed by blocking with 5% BSA in TBST for 1 h and incubation with mouse anti-HER2, HER3, phospho-HER2, phosphor-HER3 and β-actin, at 4° C. overnight. The membranes were washed in TBST 3 times, followed by incubation with HRP-anti-mouse or antibodies diluted in 5% BSA in TBST for 1 h at room temperature. The chemiluminescent signal was developed using EZ-ECL Kit (Biological Industries, Beit Haemek, Israel), and visualized by Amersham Imager 6000 (GE Healthcare UK Limited, Buckinghamshire, UK). Receptor phosphorylation was first normalized to β-actin and the receptor signals, and thereafter to divided by signal of control cells (−Nb46, −NRG).

Protein crystallization, diffraction and structure determination—Nb46 (8.5 mg/mL) were mixed at a 1:1 (v/v) ratio with a reservoir solution and crystallized, at room temperature, by the sitting-drop vapor diffusion method over a reservoir containing 17% Polyethylene Glycol 3350 and 0.15M Sodium Nitrate. The crystals were then harvested and flash-cooled in liquid nitrogen. X-ray diffraction data were collected at beamline 103 in Diamond Light Source (DLS) Didcot, UK. Data were collected at 100 K from one crystal that diffracted to a maximum resolution of 1.62 Å. The NB46 crystal belongs to the space group C2221, with unit cell dimensions of a=90.31 Å, b=107.29 Å, and c=113.05 Å, and it contains four copies of the protein in the asymmetric unit. X-ray data were merged and scaled using Aimless from CCP4 cloud and solved by molecular replacement using Phaser in CCP4 cloud. Ensemble of models which was created using CCP4 cloud was used as a search model. Refinement included alternating cycles of manual rebuilding in COOT and automated refinement using Refmac5from CCP4 cloud.

### Example 1. Preparation and Selection of Anti-HER2 Nanobodies

### Sorting Stable Dubca Cells

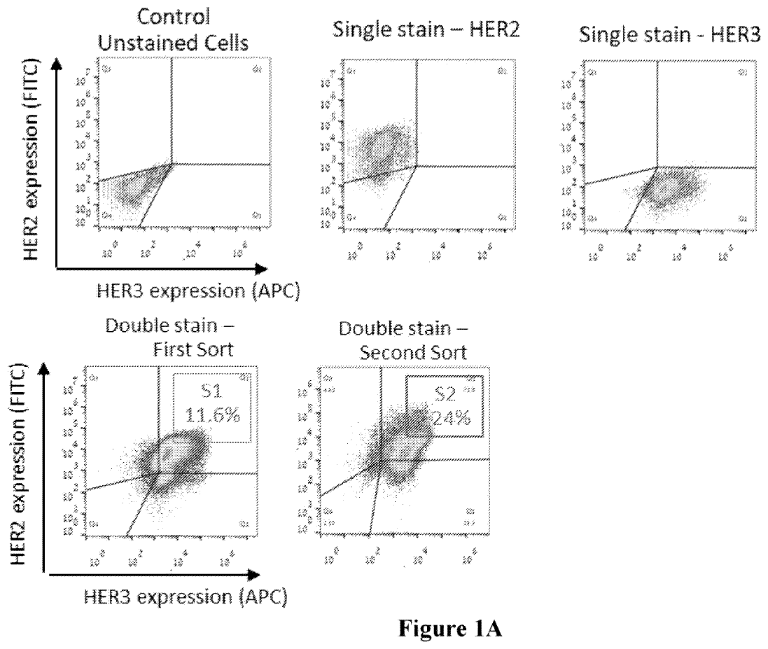

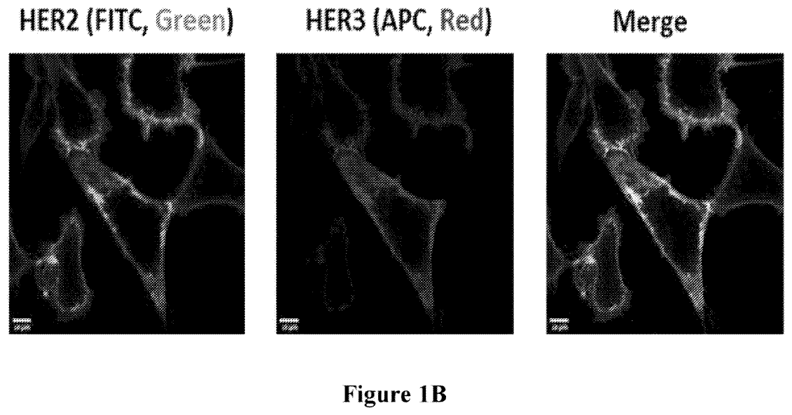

To generate camelid-nanobodies directed against the human cell membrane receptors HER2/HER3 cells were infected by lentiviruses to generate stable antigen-overexpressing cells. For the immunization, Dubca cells from the origin species of Camelus dromedaries (Arabian camel) were chosen as cell background to bias the response toward HER2/3 and to avoid immunization against other cell surface markers that exist in other common cells used for immunization, such as human HEK293 or hamster CHO cells. HER2/3-infected cells were FACS sorted for high HER2/3-expressing population in two rounds, using the BD Aria FACS (FIG. 1A). Since both lentiviruses for HER2 and HER3 hold puromycin resistance selection marker, a sorting step was used for selecting double positive population. After the first sort, the expression level of HER2 was higher than HER3 in the stable HER2/3-Dubca cells. Therefore, a second sort was performed to achieve a population with similar expression level for both receptors. The HER2/3 stable cells revealed co-localization of both HER2 and HER3 receptors in a confocal microscope image (FIG. 1B).

### Immune Monitoring of the Camels

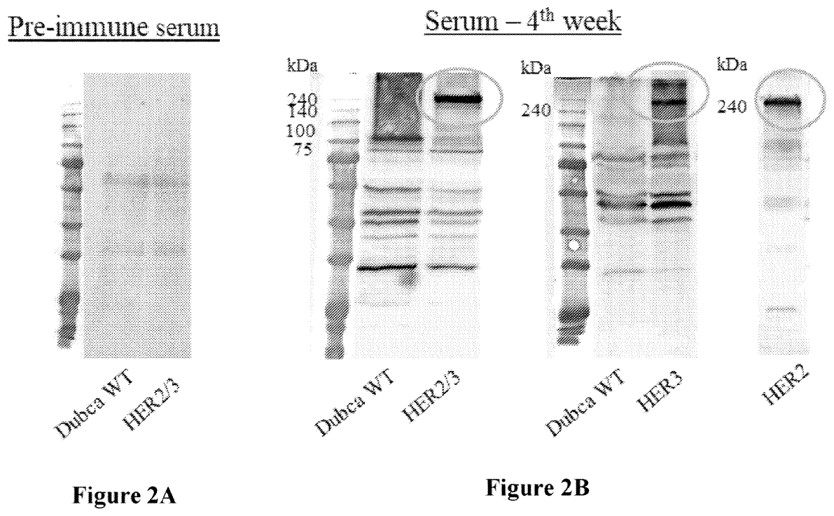

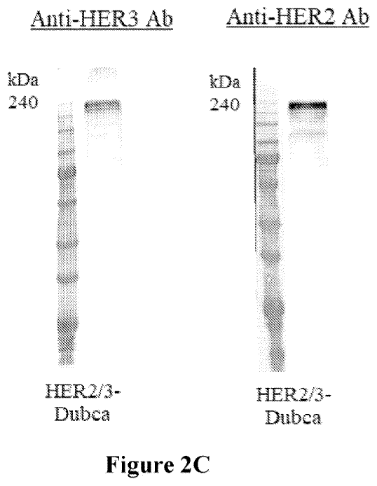

A female Camel was immunized 8 times at weekly intervals with HER2/3 expressing Dubca cells. Three days after the fourth injection, a small test bleed was taken from the camel, and the serum was tested to detect an immune response against human HER2 and HER3 receptors. The presence of HER2/3 specific nanobodies was assayed by western blot (WB) using the serum obtained at pre-immune and 4-week post-immune time points (FIG. 2). The pre-immune serum was used as a negative control and was taken from the camel prior to the first injection, and as expected no specific response was detected against lysates of Dubca wildtype (WT, uninfected control), and HER2/3 expressing cells. The membranes were incubated with the serum followed by incubation with anti-camelid VHH-HRP Ab (FIG. 2A). The faint bands were a result of a 5 min long exposure and appear similar for all samples and marking them as non-specific. However, a specific response was observed in the membranes incubated with serum of an immunized camel (from week 4 post immunization, FIG. 2B). The specific distinct bands at 240 kDa correspond to serum nanobody response detecting HER2/3, HER2, and HER3 (circled) in comparison with Dubca WT. Other lower bands on these membranes are background as they are visible also in the Dubca WT lanes. To validate that the observed bands represent the human antigens HER2 and HER3 the membranes that were blotted with HER2/3-expressed cells, were incubated with specific anti-human HER3 Ab or anti-human HER2 Ab. The new observed bands were of the same size, corresponding to a molecular weight of ˜240 kDa (FIG. 2C).

### Construction of the Nanobody Library

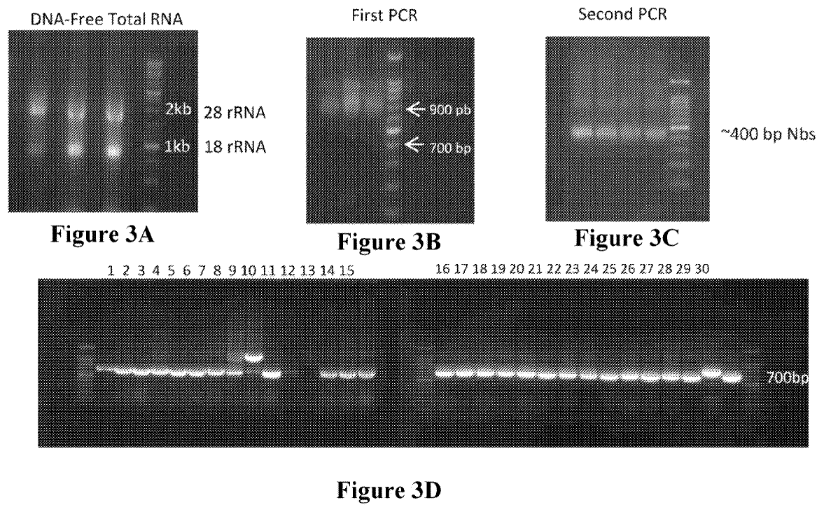

At the end of the immunization series, whole blood was collected from the camel for the isolation of lymphocyte RNA (FIG. 3A), that was reverse-transcribed into cDNA, from which the Nbs library was amplified in a two-step-PCR. The first PCR amplification resulted in two products, i.e., a ˜900 bp fragment for VH-CH1-CH2 fragment and ˜700 bp for VHH-CH2 fragment (FIG. 3B). The second PCR used the 700 bp fragments as template to amplify a ˜400 bp fragments corresponding to the Nb size (FIG. 3C), which were cloned into a phage-display vector pMECS. A total of 24 electroporation reactions were performed to transform the vector into TG1 E. coli cells, to obtain a high-quality library with great diversity. The nanobody library size of 2.0×10⁹ colonies was calculated from the number of independent colonies on LB-agar plates. Colony PCR analysis (with primers MP57 and GIII) on 30 random colonies revealed that the percentage of colonies with vector containing a nanobody insert of the proper size reached 90% (FIG. 3D).

### Periplasmic-ELISA Screen for High-Affinity Nanobodies

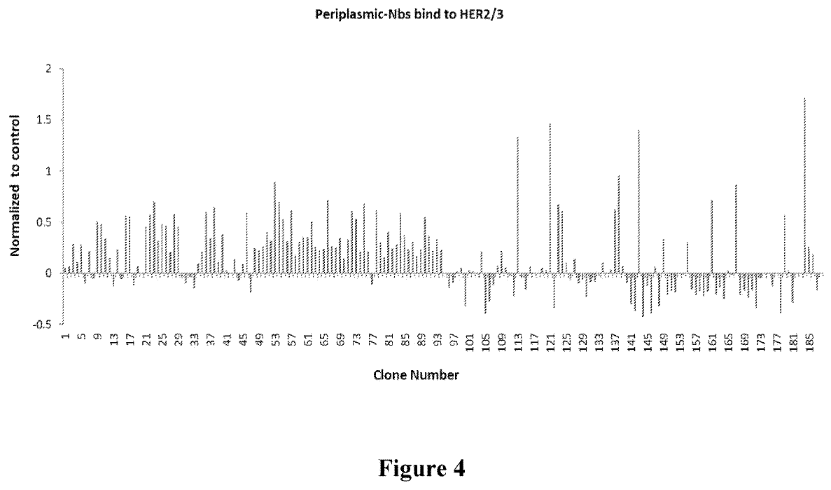

A cell-based panning process was repeated five times using phage-display with stable Dubca cells expressing HER2/3. After each panning round, Nb-displaying phages were transferred into naive TG1 E. coli, which were plated on LB-ampicillin glucose plates for backup. Individual 188 bacteria colonies were picked from LB plates (from the fifth panning round) and cultured to isolate the Nbs as proteins from the bacteria periplasm. These periplasmic-Nbs were screened by ELISA to identify variants that had the best binding to HER2/3 expressing cells (FIG. 4). Dubca WT cells were used as a background control and the OD₄₅₀ absorbance value of periplasmic-Nbs binding to HER2/3 was normalized to that from the binding of periplasmic-Nbs to WT Dubca (Y-axis). Of the 188 periplasmic-Nbs clones 28 showed good target recognition. These Nb clones (originated from TG1 colonies) were further amplified by PCR and sequenced.

### Selection of Nanobody Clones

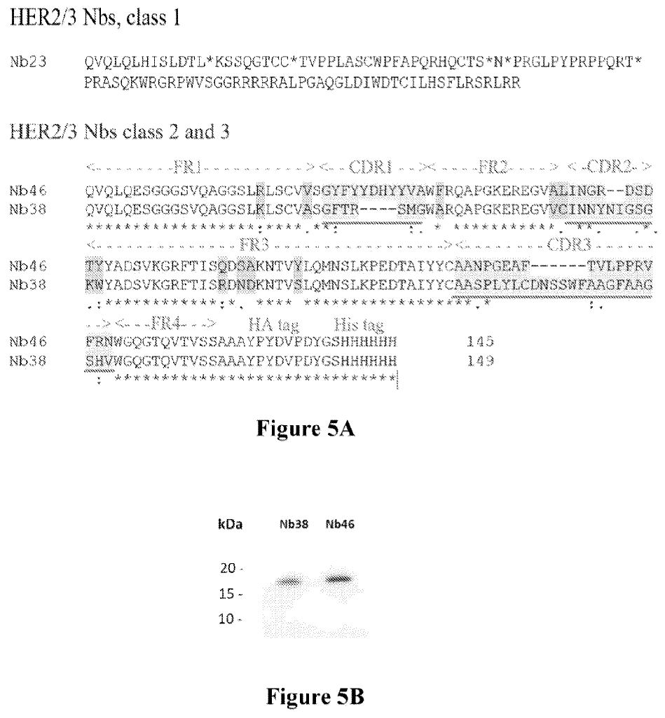

Based on sequencing data of 28 HER2/3 Nb clones, 8 had an early stop codon that also appeared in multiple places throughout the sequence, and 20 contained the correct (in frame) Nb fragments. They were classified into 3 classes based on sequence identity, which are shown by diversified framework region (FR), and complementarity determining regions (CDRs) sequences (FIG. 5A). 3 classes of nanobodies were identified as Nb23, Nb38, and Nb46.

In order to further express nanobodies in E. coli, the clones for Nb38, Nb46, and Nb23 in phagemid pMECS were transformed into WK6 E. coli cells to express and purify nanobodies in a large scale (FIG. 5B). In WK6, the expressed Nbs are secreted to the periplasm compartment, where the oxidizing conditions favor the formation of the disulfide bonds that stabilizes the Nb structure. Extraction of the Nbs is done by compromising the bacteria outer membrane wall, by osmotic shock, without the need to lyse the cells. SDS-PAGE gel analysis demonstrated the good quality of Nbs obtained with high purity and expected size of ˜16 kDa. The Nbs purification process resulted in a yield of ˜1-1.8 mg/L culture.

### Example 2. Binding Analysis of Nanobodies Against HER2/3 Expressing Cells

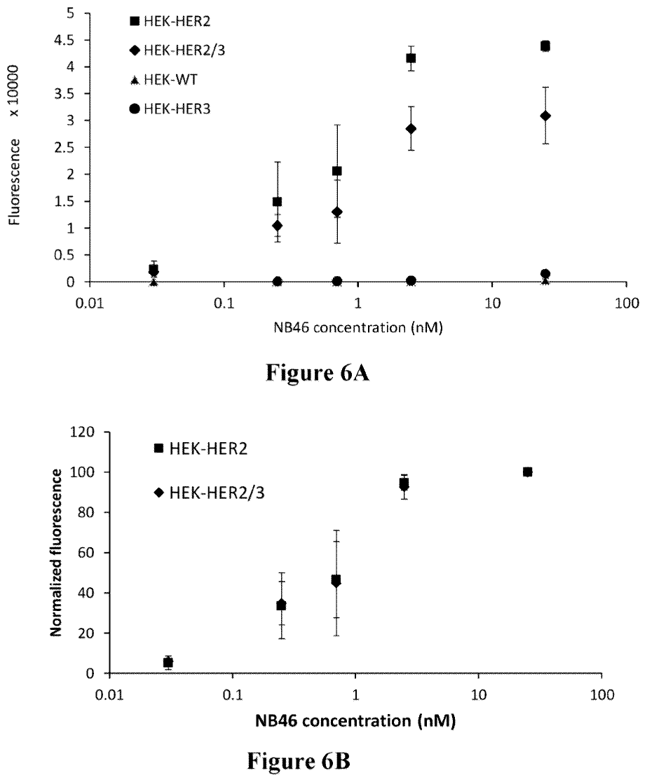

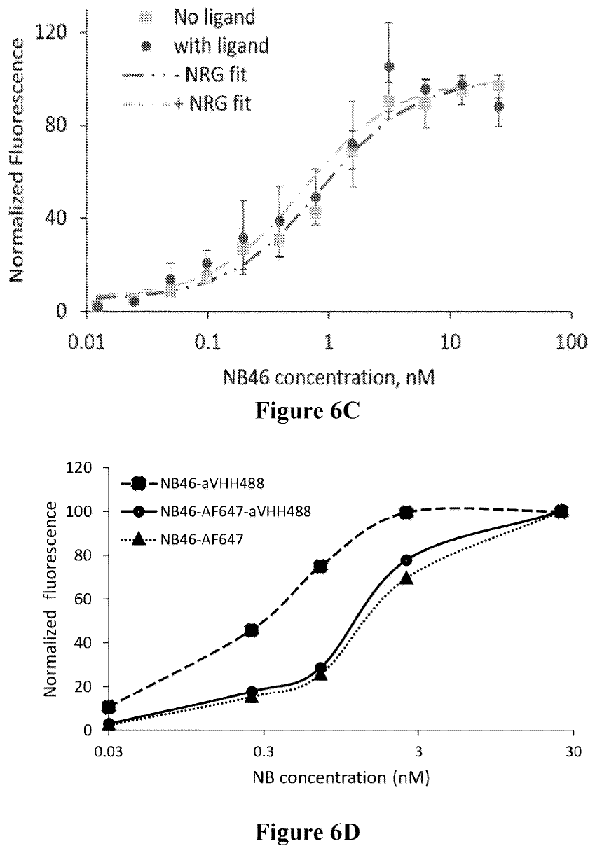

The Nb46 showed binding of HEK cells expressing HER2 and both HER2 and HER3 (HER2/3) receptors, but not to HER3 expressing cells, versus control of WT cells (FIGS. 6A and 6B). The normalized fluorescent signals overlap between cells expressing HER2 and HER2/3, which indicates that Nb46 binds to a target found on HER2 receptor which is uninterrupted by possible dimerization with HER3 (FIG. 6B). Titration of analysis of SkBr3, HER2+ breast cancer cells with Nb46, reveals that Nb46 binds with a high affinity of KD=0.8 nM, and that the binding is hardly affected by Neuregulin-1 (NRG, ligand for HER3). As interaction of NRG with HER3 activates the receptor towards forming heterodimers with HER2, the lack of increased observed signal with NRG suggests that the binding target for Nb46 is outside of the HER2-HER3 dimerization site (FIG. 6C). Fluorescently labelled Nb46 (NB46-AF647, labelled in-house with approximately 1 mole dye/mole NB) showed similar binding of SkBr3 cells as of the unlabelled NB46 (FIG. 6D). The Nb38 failed to show binding of either HER2, HER3 and cells co-expressing both, and thus excluded from further analysis.

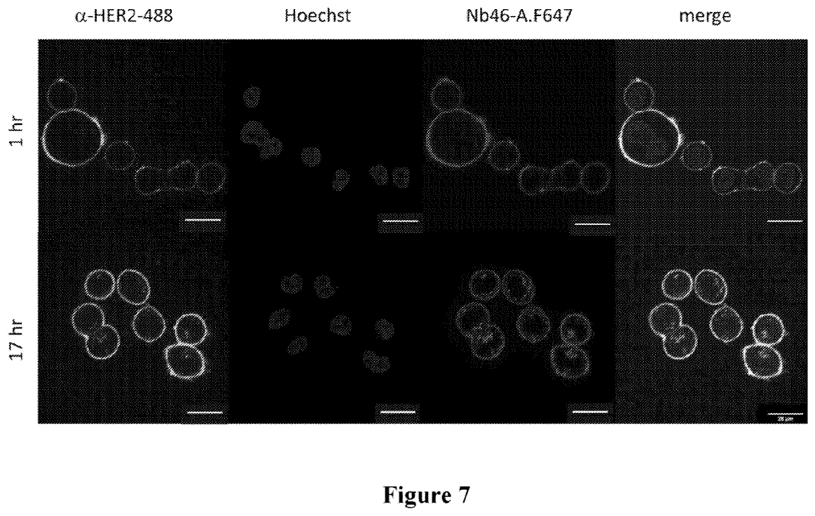

NB46-AF647 binding of the cells membranes is apparent by colocalization with HER2-receptor on the cell membrane (FIG. 7, merge of top row). Subsequent internalization was viewed following 17 h of incubation by depletion of signal from the membranes and emergence of red stain within the cell's cytoplasm (FIG. 7, bottom row).

### Example 3. Binding Analysis of Nb46 Against Recombinant HER2 by SPR

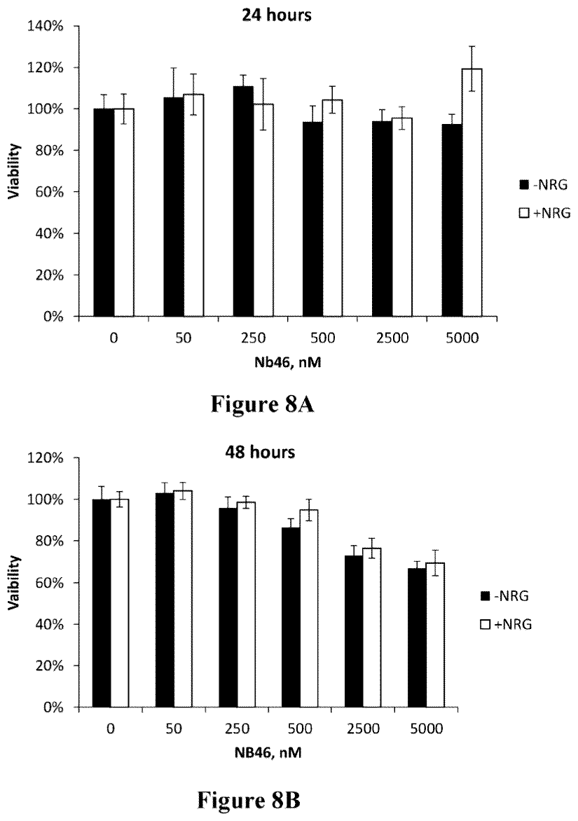

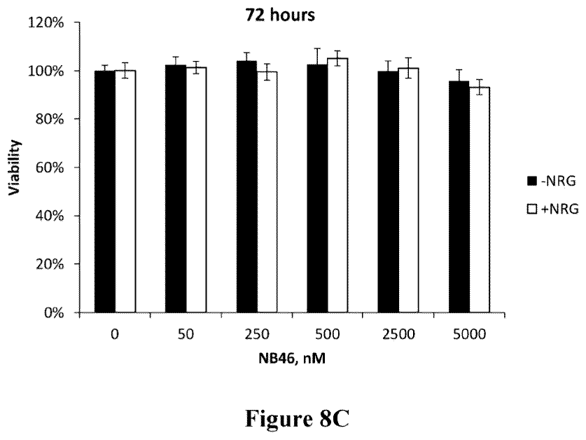

SPR revealed that the binding affinity of purified Nb46 to recombinant HER2 is of sub-nanomolar magnitude. Similar dissociation constants of K_{D}=0.24±0.07 nM and K_{D}=0.17±0.09 nM (average: 0.21±0.08 nM) were measured when the HER receptor was immobilized and the Nb was pumped as the analyte and vice versa, respectively (FIG. 8). The kinetic parameters are shown in Table 3. Nb46 showed no detectable binding towards HER3 in either analyte-ligand configuration. Thermal stability assay of Nb46 using a melting curve profile of 0.3 mg/mL NB46 revealed a Tm of 76° C.

TABLE 3
Nb46 binding of HER2 - kinetic parameters

| Ligand | Analyte | ka (1/Ms) · 10⁶ | kd (1/s) · 10⁻³ |
|---|---|---|---|
| Nb46 | HER2 | 12.6 ± 0.1 | 2.21 ± 0.01 |
| HER2 | Nb46 | 6.26 ± 0.04 | 1.54 ± 0.01 |
| | Average | 9.42 ± 0.08 | 1.87 ± 0.01 |

### Example 4. Cells Viability and HER2,3 Receptors Phosphorylation after Binding Nb46

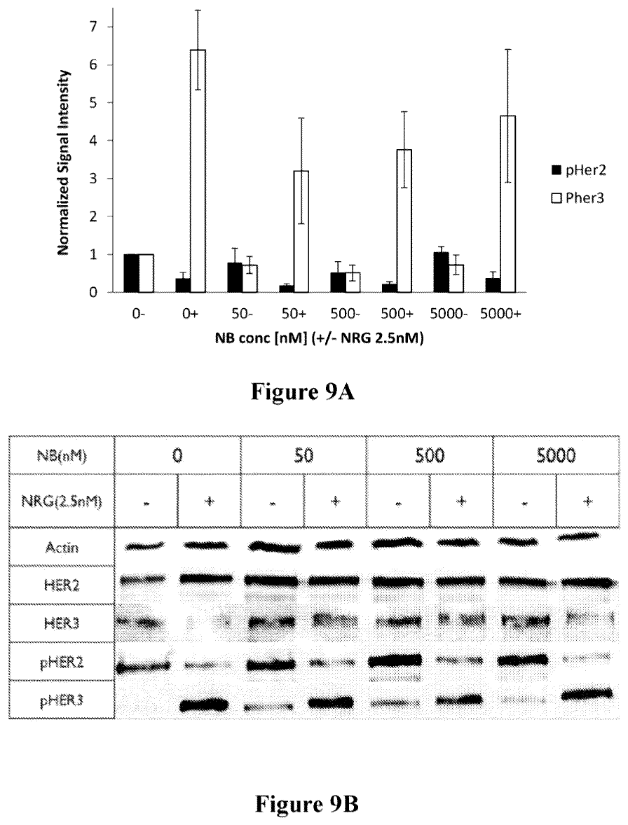

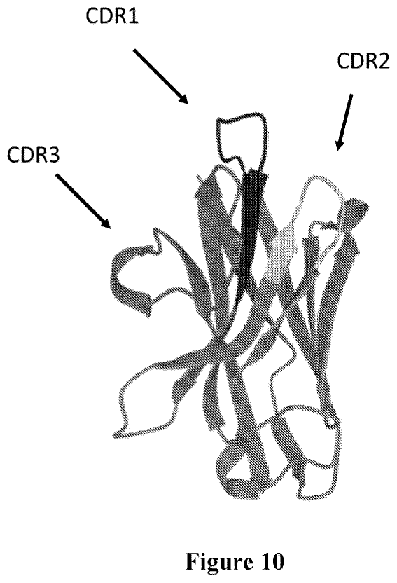

The effects of Nb46 on the cellular functions of SkBr3 cells, following the binding of the Nb to its target receptor was determined. The Nb has a neglectable effect on SkBr3 cells viability, regardless of NRG presence after 24 and 72 h incubation (with exception for 5000 nM Nb46, without NRG, last gray bar at 24 h, FIG. 8). After 48 h incubation the cells viability seems to decrease with increase in Nb46 concentration, starting from 250 nM compared to untreated cells. Observation of an effect on 48 h may indicate that Nb46 influences cell division, as the effect is noticeable only the 37 h doubling time, characteristic of these cells. The effect on receptors phosphorylation after short term exposure to Nb46 was tested by western blot, using antibodies targeting the phosphorylation site on HER2 and HER3 (FIG. 9). The results show an expected increase in HER3 phosphorylation in samples containing NRG, compared with those w/o NRG, in all tested Nb46 concentrations. The lack of influence of Nb46 on HER3 phosphorylation may indicate that the binding of Nb46 is outside of the HER2 dimerization site. The solved crystal structure of Nb46 is shown in FIG. 10.

### Example 5. Nb46 Shows No Effect on HERs Recycling

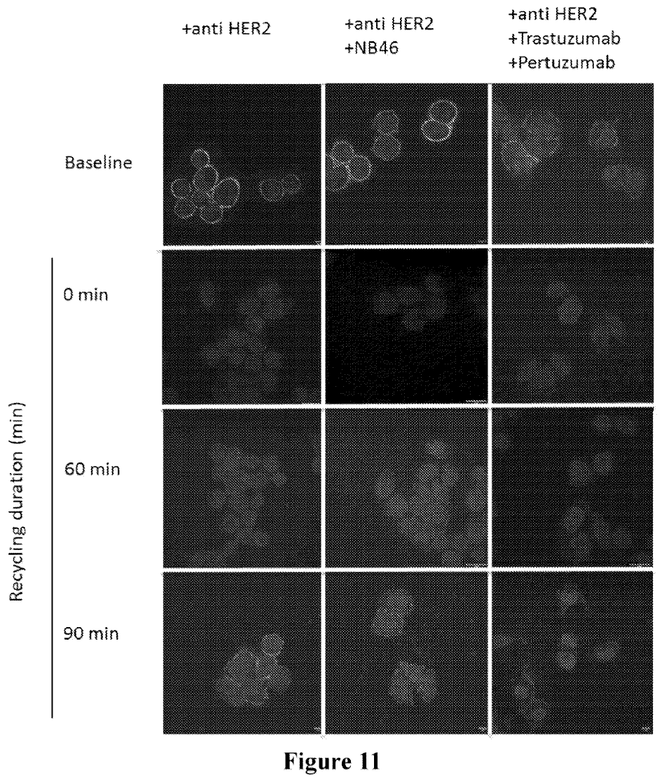

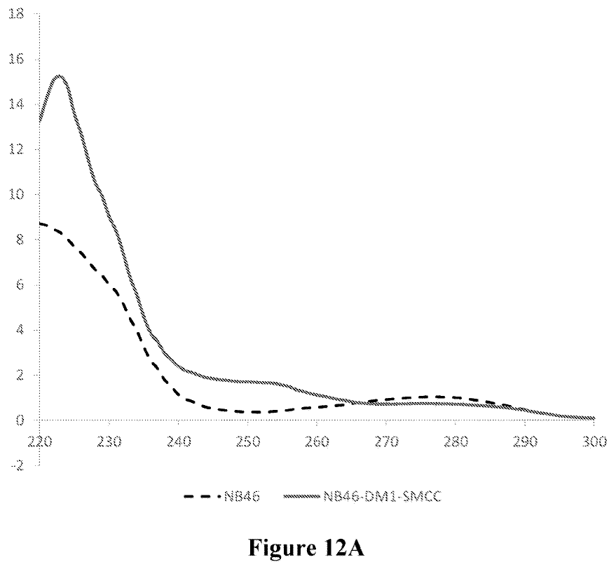

In order to examine the recycling of HER2 receptor, cell surface HER2 receptors and nucleus at SKBR3 cells were pre-labeled with anti-HER2 antibody (FITC) and Hoechst 33342. Some of the cells were also pre-labeled with 1 nM NB46 or with 1 mg/ml Trastuzumab and Pertuzumab (4° C.). Cells were either fixed without inducing endocytosis (Baseline) or induced to undergo endocytosis by incubation at 37° C. After stripping any anti-HER2 antibody, NB46, Trastuzumab and Pertuzumab remaining on the cell surface with an acid wash at 4° C. after the pulse of endocytosis, HER2 recycling was initiated by incubation at 37° C. and allowed to continue for 90 min. The cells were imaged on a 3i Marianas (Denver, CO) spinning disk confocal microscope equipped with Yokogawa W1 module and Prime 95B sCMOS camera. As shown in FIG. 11, NB46 had no effect on HER2 receptor recycling.

Trastuzumab combined with Pertuzumab induces HER2 internalization and intracellular degradation. This corresponds to the results obtained in FIG. 11. After 60 and 90 minutes, there is no recycling of HER2 receptor to the membrane at the Trastuzumab combined with Pertuzumab treatment wells. The reduction of HER2 expression in breast cancer cells can lead to the development of acquired resistance to HER2-targeted therapies such as Trastuzumab and Pertuzumab. This is a significant clinical challenge that can limit the effectiveness of these therapies and lead to disease progression. On the other hand, during the NB46 treatment, the HER2 receptor recycles to the membrane after 90 minutes. These results suggest that NB46 will overcome or prevent the development of resistance in HER2 positive breast cancer.

### Example 5. Nb46 is Suitable for Use in Antibody Drug Conjugate (ADC)

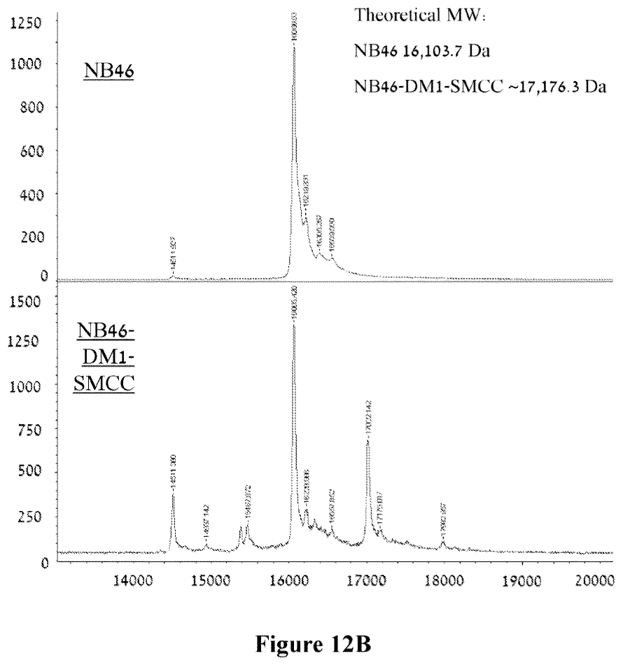

DM1-SMCC (1072.6 Da) is a drug-linker conjugate composed of a potent microtubule-disrupting agent DM1 and a linker SMCC to make antibody drug conjugate (ADC). NB46 (1.09 mg) in conjugation buffer (PBS, PH 8.9) conjugated with SMCC-DM1 (Amadis Chemicals, Zhejiang, China) at room temperature with a molar ratio of 1:10 (in 20% DMF) for 1.5 hours. The mixture was centrifuged at 21,000 g for 20 min to remove aggregates. Then the mixture was centrifuged with AMICON Ultra (3 kDA, Merck, NJ, USA) to remove the unconjugated DM1-SMCC and organic solvent. The concentration of the ADC was measured by Nanodrop spectrophotometer and BCA protein assay kit (Pierce, Waltham, USA). The ADC were analyzed using MALDI-TOF MS (matrix-assisted laser desorption ionization time-of-flight mass spectrometry). Also, SDS-Page analysis (Gel contain 20% Acrylamide) were conducted with the ADC and the unconjugated NB46. There are differences in the absorbance of the NB46 against NB46-DM1-SMCC in the Nanodrop (FIG. 12A). The absorbance spectrum of the NB46 shows a peak at 220 and 280 nm, the peak at 280 nm is characteristic of the absorption of aromatic amino acids, particularly tryptophan. The absorbance spectrum of DM1-SMCC usually has two main peaks, at around 250-260 nm. The aromatic ring of DM1 (maytansine) attributes the 250-260 nm peak. The absorbance spectrum of the ADC NB46-DM1-SMCC shows a 280 nm peak and additional peaks at about 220-230 and shoulder at about 250-260 nm. There are also differences in the spectra of the NB46 against NB46-DM1-SMCC in the MALDI-TOF (FIG. 12B). NB46's theoretical molecular weight is 16,103.7 Da, and the measured one is 16066.5 Da (37 Da difference). NB46-DM1-SMCC's theoretical molecular weight is about 17,176.3 Da, and the measured one is 17,022.1 Da (154 Da difference). In the NB46-DM1-SMCC, there is also peak for non-conjugated NB46 at 16,065.4 (38 Da difference). That is considering the efficiency of the process as not all the NB46 conjugated.

Sequences:

Nb46 VHH-Amino acid sequence (SEQ ID NO: 1)
QVQLQESGGGSVQAGGSLRLSCVVSGYFYYDHYYVAWFRQAPGKEREGV
ALINGRDSDTYYADSVKGRFTISQDSAKNTVYLQMNSLKPEDTAIYYCA
ANPGEAFTVLPPRVFRNWGQGTQVTVSS
Nb46 VHH CDR1 (SEQ ID NO: 2)
GYFYYDHYYVA
Nb46 VHH CDR2 (SEQ ID NO: 3)
INGRDSD
Nb46 VHH CDR3 (SEQ ID NO: 4)
AANPGEAFTVLPPRVFRN
Nb38 VHH-Amino acid sequence (SEQ ID NO: 5)
QVQLQESGGGSVQAGGSLKLSCVASGFTRSMGWARQAPGKEREGVVCIN
NYNIGSGKWYADSVKGRFTISRDNDKNTVSLQMNSLKPEDTAIYYCAAS
PLYLCDNSSWFAAGFAAGSHVWGQGTQVTVSS
Nb38 VHH CDR1 (SEQ ID NO: 6)
GFTRSMG
Nb38 VHH CDR2 (SEQ ID NO: 7)
INNYNIGSG
Nb38 VHH CDR3 (SEQ ID NO: 8)
AASPLYLCDNSSWFAAGFAAGSHV
HA-tag + His-tag amino acid sequence
(SEQ ID NO: 9)
AAAYPYDVPDYGSHHHHHH

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

1. An anti-HER2 binding molecule or a fragment, derivative or analog thereof, the binding molecule comprising a set of three CDR sequences wherein the set is selected from the group consisting of:
(i) a set derived from VHH termed Nb46 comprising the CDR sequences: GYFYYDHYYVA (SEQ ID NO: 2), INGRDSD (Sequence No: 3) and AANPGEAFTVLPPRVFRN (SEQ ID NO: 4); and
(ii) a set derived from VHH termed Nb38 comprising the CDR sequences: GFTRSMG (SEQ ID NO: 6), INNYNIGSG (SEQ ID NO: 7), and AASPLYLCDNSSWFAAGFAAGSHV (SEQ ID NO: 8).

2. The binding molecule of claim 1, comprising an amino acid sequence at least about 90% identical to a sequence selected from the group consisting of SEQ ID Nos: 1 and 5.

3. The binding molecule of claim 1, wherein the binding molecule is a single chain antibody.

4. The binding molecule of claim 1, wherein the binding molecule is a heavy chain single-domain (VHH) antibody.

5. The binding molecule of claim 1, wherein the binding molecule is a camelid antibody.

6. The binding molecule of claim 1, wherein the fragment comprises an antigen binding domain.

7. (canceled)

8. The binding molecule of claim 1, wherein the binding molecule binds to the HER2 protein with an affinity of at least 10⁻⁸M.

9. The binding molecule of claim 1, wherein the binding molecule is characterized by molecular weight of less than 30 kDa.

10. A fusion protein comprising the binding molecule of claim 1 and a tag.

11. (canceled)

12. (canceled)

13. A conjugate comprising the anti-HER2 binding molecule or fragment according to claim 1.

14. The conjugate of claim 13, wherein the binding molecule is attached to a cytotoxic moiety, a radioactive moiety, or an affinity or labeling tag.

15. The conjugate of claim 13, wherein the binding molecule is attached to a toxin selected from the group consisting of microtubule inhibitor, DNA synthesis inhibitor, topoisomerase inhibitor and RNA polymerase inhibitor.

16. The conjugate of claim 15, wherein the microtubule inhibitor is DM1.

17. A polynucleotide encoding the binding molecule according to claim 1.

18. A cell capable of producing an at least one binding molecule according to claim 1.

19. A pharmaceutical composition comprising at least one binding molecule according to claim 1, and a pharmaceutically acceptable excipient, carrier, or diluent.

20. (canceled)

21. (canceled)

22. (canceled)

23. (canceled)

24. A method of treating cancer comprising administering to a subject in need thereof, a therapeutically effective amount of at least one binding molecule according to claim 1.

25. The method of claim 24, wherein the cancer is HER2+ cancer.

26. (canceled)

27. (canceled)

28. (canceled)

29. (canceled)

30. (canceled)

31. A method of diagnosing or prognosing HER2+ cancer in a subject, the method comprises determining the expression level of HER2 in a biological sample of said subject using at least one binding molecule or fragment according to claim 1.

32. A method of determining or quantifying the expression of HER2, the method comprising contacting a biological sample with a binding molecule or fragment according to claim 1.

33. (canceled)


SEQUENCE LISTING (15 sequences)

SEQ ID NO: 1 - protein, 126 aa, unidentified
QVQLQESGGGSVQAGGSLRLSCVVSGYFYYDHYYVAWFRQAPGKEREGVALINGRDSDTYYADSVKGRFTISQDSAKNTVYLQMNSLKPEDTAIYYCAANPGEAFTVLPPRVFRNWGQGTQVTVSS

SEQ ID NO: 2 - protein, 11 aa, unidentified
GYFYYDHYYVA

SEQ ID NO: 3 - protein, 7 aa, unidentified
INGRDSD

SEQ ID NO: 4 - protein, 18 aa, unidentified
AANPGEAFTVLPPRVFRN

SEQ ID NO: 5 - protein, 130 aa, unidentified
QVQLQESGGGSVQAGGSLKLSCVASGFTRSMGWARQAPGKEREGVVCINNYNIGSGKWYADSVKGRFTISRDNDKNTVSLQMNSLKPEDTAIYYCAASPLYLCDNSSWFAAGFAAGSHVWGQGTQVTVSS

SEQ ID NO: 6 - protein, 7 aa, unidentified
GFTRSMG

SEQ ID NO: 7 - protein, 9 aa, unidentified
INNYNIGSG

SEQ ID NO: 8 - protein, 24 aa, unidentified
AASPLYLCDNSSWFAAGFAAGSHV

SEQ ID NO: 9 - protein, 19 aa, unidentified
AAAYPYDVPDYGSHHHHHH

SEQ ID NO: 10 - DNA, 23 bp, unidentified
  source 1..23: mol_type = other DNA
gtcctggctgctcttctacaagg

SEQ ID NO: 11 - DNA, 23 bp, unidentified
  source 1..23: mol_type = other DNA
ggtacgtgctgttgaactgttcc

SEQ ID NO: 12 - DNA, 34 bp, unidentified
  source 1..34: mol_type = other DNA
ctagtgcggccgctgaggagacggtgacctgggt

SEQ ID NO: 13 - DNA, 29 bp, unidentified
  source 1..29: mol_type = other DNA
gatgtgcagctgcaggagtctggaggagg

SEQ ID NO: 14 - DNA, 20 bp, unidentified
  source 1..20: mol_type = other DNA
ttatgcttccggctcgtatg

SEQ ID NO: 15 - DNA, 19 bp, unidentified
  source 1..19: mol_type = other DNA
ccacagacagccctcatag